(19) United States
(12) Patent Application Publication
(10) Pub. No.: US 2002/0197234 A1
(43) Pub. Date: Dec. 26, 2002

(54) Immunologic activities of rhesus cytomegalovirus encoded IL-10 and human cytomegalovirus encoded IL-10

(75) Inventors: Thomas J. Schall, Menlo Park, CA (US); Mark Penfold, Mountain View, CA (US); Juliet Spencer, Foster City, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ChemoCentryx, Inc., San Carlos, CA (US)

(21) Appl. No.: 09/919,224

(22) Filed: Jul. 30, 2001

Related U.S. Application Data
(60) Provisional application No. 60/221,831, filed on Jul. 28, 2000.

(51) Int. Cl. A61K 38/20; A61K 45/00; C12N 5/08

(52) U.S. Cl. 424/85.2; 424/93.7; 435/372

(57) ABSTRACT

The present invention is provides pharmaceutical compositions and prophylactic and therapeutic methods of treatment for immune disorders using rhesus or human CMV IL-10. The invention is useful for inhibiting lymphocyte proliferation and underlying cellular events both in vitro and in vivo.

### CROSS REFERENCE TO RELATED APPLICATION

This application is a nonprovisional of U.S. application No. 60/221,831, filed Jul. 28, 2000, which is incorporated herein by reference in its entirety for all purposes.

### BACKGROUND OF THE INVENTION

Cytomegaloviruses (CMVs) are members of the beta subgroup of the herpesvirus family. CMV is a slow replicating, species-specific complex DNA virus found in most mammals. The CMV phenotype is distinguished by slow replication in a limited number of cell types and a typical cytopathology. Human CMV has a 230-kb double stranded DNA genome encoding at least 200 open reading frames (ORFs), giving CMV the highest potential coding capacity within the herpesvirus family. A viral IL-10-like protein encoded by ORF UL111A has been identified within the human CMV genome. The ORF corresponding to the UL111A ORF of human CMV has been identified in rhesus CMV genome and encodes for a viral IL-10-like protein.

### BRIEF DESCRIPTION OF THE DRAWINGS

FIG. 1 shows a western blot of supernatants from HEK293 cells transfected with control vector pcDNA3.1-m/H, expression vector pcDNA3.1 m/H-rhesus CMV IL-10, and expression vector C-terminal myc/His epitope-tagged human CMV IL-10.

FIGS. 2A, 2B and 2C. FIG. 2A is a graphic representation of the proliferation of rhesus PBMCs in the presence of either mock supernatants, rhesus CMV IL-10 containing supernatants or supernatants depleted of rhesus CMV IL-10 by immunoprecipitation. FIG. 2B is a graphic representation of the proliferation of human PBMCs in the presence of either mock supernatants, rhesus CMV IL-10 containing supernatants or supernatants depleted of rhesus CMV IL-10 by immunoprecipitation. FIG. 2C is a graphic representation of the proliferation of human PBMCs in the presence of either mock supernatant, human CMV IL-10 containing supernatant or rhesus IL-10 treat supernatant.

FIGS. 3A and 3B. FIG. 3A shows the response of human PBMCs dosed with rhesus CMV IL-10 or rhIL-10. FIG. 3B shows the response of human PBMCs dosed with human CMV IL-10 or hIL-10.

FIG. 4 shows the rhesus CMV IL-10 dose dependent inhibition of PBMC proliferation.

FIGS. 5A, 5B, and 5C. FIG. 5A is a graphic representation of IFN-γ production by human PBMCs treated with rhesus CMV IL-10 or hIL-10. FIG. 5B is a graphic representation of IFN-γ production by rhesus PBMCs treated with human CMV IL-10 or rhIL-10. FIG. 5C is a graphic representation of IFN-γ production by human PBMCs treated with rhesus CMV IL-10, human CMV IL-10, or human IL-10.

FIGS. 6A and 6B. FIG. 6A is a graphic representation of TNF-α production by human PBMCs treated with rhesus CMV IL-10 or hIL-10. FIG. 6B is a graphic representation of TNF-α production by rhesus PBMCs treated with human CMV IL-10 or hIL-10.

FIGS. 7A, 7B, 7C, and 7D. FIG. 7A is a graphic representation of IL-1α production by human monocytes treated with rhesus CMV IL-10 or rhIL-10. FIG. 7B is a graphic representation of GM-CSF production by human monocytes treated with rhesus CMV IL-10 or rhIL-10. FIG. 7C is a graphic representation of TNF-α production by human monocytes treated with rhesus CMV IL-10 or rhIL-10. FIG. 7D is a graphic representation of IL-6 production by human monocytes treated with rhesus CMV IL-10 or rhIL-10.

FIGS. 8A, 8B, 8C, and 8D. FIG. 8A is a graphic representation of TNF-α production by human monocytes treated with human CMV IL-10 or rhIL-10. FIG. 8B is a graphic representation of GM-CSF production by human monocytes treated with human CMV IL-10 or rhIL-10. FIG. 8C is a graphic representation of IL-1α production by human monocytes treated with human CMV IL-10 or rhIL-10. FIG. 8D is a graphic representation of IL-6 production by human monocytes treated with rhesus CMV IL-10 or rhIL-10.

FIG. 9 is a graphic representation of the surface expression of CD54, classical class I MHC, class II MHC and HLA-G molecules by monocytes treated with rhesus CMV IL-10.

FIG. 10 is a graphic representation of the surface expression of CD54, classical class I MHC, class II MHC and HLA-G molecules by monocytes treated with human CMV IL-10.

FIGS. 11A and 11B. FIG. 11A is a graphic representation of the inhibition of proliferation of human PBMCs treated with recombinant human IL-10. FIG. 11B is a graphic representation of the inhibition of proliferation human PBMCs treated with recombinant human CMV IL-10.

FIGS. 12A and 12B. FIG. 12A is a graphic representation of the activity of recombinant human IL-10 neutralized by an antibody to the human IL-10 receptor. FIG. 12B is a graphic representation of the activity of recombinant human CMV IL-10 neutralized by an antibody to the human IL-10 receptor.

### DETAILED DESCRIPTION OF THE INVENTION

I. Definitions

For purposes of the present invention, the following terms are defined below:

The term “allergen” means noninfectious antigens that induce hypersensitivity reactions, most commonly IgE-mediated type I reaction.

The term “allergy” means a type I hypersensitivity reaction that can include hay fever, asthma, serum sickness, systemic anaphylaxis or contact dermatitis

The term “allogeneic” denotes members of the same species that differ genetically. “Alloantigen” means antigenic determinates present on molecules that different among members of the same species.

The term “antibody” refers to intact antibodies and binding fragments thereof. Typically, fragments compete with the intact antibody from which they were derived and with other antibodies for specific binding to an antigen

The term “antigen presenting cell (APC)” means any cell that can process and present antigenic peptides in association with class II MHC molecules and deliver a co-stimulatory signal necessary for T-cell activation.

The term “autoimmune disease” refers to a spontaneous or induced malfunction of the immune system of mammals in which the immune system fails to distinguish between foreign immunogenic substances within the mammal and/or autologous (“self”) substances and, as a result, treats autologous (“self”) tissues and substances as if they were foreign and mounts an immune response against them. Autoimmune disease is characterized by production of either antibodies that react with self tissue, and/or the activation of immune effector T cells that are autoreactive to endogenous self antigens. Three main immunopathologic mechanisms act to mediate autoimmune diseases: 1) autoantibodies are directed against functional cellular receptors or other cell surface molecules, and either stimulate or inhibit specialized cellular function with or without destruction of cells or tissues; 2) autoantigen—autoantibody immune complexes form in intercellular fluids or in the general circulation and ultimately mediate tissue damage; and 3) lymphocytes produce tissue lesions by release of cytokines or by attracting other destructive inflammatory cell types to the lesions. These inflammatory cells in turn lead to production of lipid mediators and cytokines with associated inflammatory disease.

The term “ELISA (enzyme linked immunoabsorbent assay)” means an assay for quantitating either antibody or antigen by antibody and substrate that forms a colored reaction product.

The term “gene therapy” refers to the transfer of genetic material (e.g., DNA or RNA) of interest into a host to treat or prevent a genetic or acquired disease or condition. The genetic material of interest encodes a product (e.g., a protein polypeptide, peptide or functional RNA) whose production in vivo is desired. For example, the genetic material of interest can encode a hormone, receptor, enzyme or protein or fragment thereof of therapeutic value.

The term “graft” means any tissue or organ for transplantation. An “allograft” is tissue that is transferred between genetically different members of the same species.

The term “hypersensitivity” means an exaggerated immune response that causes damage to the individual. Immediate hypersensitivity types I and II are mediated by antibody complexes. Immediate hypersensitivity type III is mediated by immune complexes. Delayed type hypersensitivity (type IV) is mediated by T_{DTH} cells. Immediate hypersensitivity usually occurs minutes to hours after exposure of a sensitized individual to an antigen. DTH usually occurs 2-3 days after the T_{DTH} cells interact with antigen.

The term “immune-mediated” refers to a process that is either autoimmune or inflammatory in nature.

The term “immunological” or “immune” response is the development of a beneficial humoral (antibody mediated) and/or a cellular (mediated by antigen-specific T cells or their secretion products) response directed against an immunogen. Such a response can be an active response induced by administration of immunogen or a passive response induced by administration of antibody or primed T-cells. A cellular immune response is elicited by the presentation of polypeptide epitopes in association with Class I or Class II MHC molecules to activate antigen-specific CD4+ T helper cells and/or CD8+ cytotoxic T cells. The response can also involve activation of monocytes, macrophages, NK cells, basophils, dendritic cells, astrocytes, microglia cells, eosinophils or other components of innate immunity. The presence of a cell-mediated immunological response can be determined by proliferation assays (CD4+ T cells) or CTL (cytotoxic T lymphocyte) assays. (See, Burke et al., (1994) J. Inf. Dis. 170, 1110-19; Tigges et al., (1996) J. Immunol. 156, 3901-3910, incorporated herein by reference). The relative contributions of humoral and cellular responses to the protective or therapeutic effect of an immunogen can be distinguished by separately isolating IgG and T-cells from an immunized syngeneic animal and measuring protective or therapeutic effect in a second subject. A useful background to the immune system is provided, for example, in Goldsby et al., (2000) Kuby Immunology (4th Ed.) W. H. Freeman and Company, New York, incorporated herein by reference. Chapters 15 on leukocyte migration and inflammation, 16 on hypersensitive reactions, 20 on autoimmunity, 21 on transplantation immunology, 22 on cancer and the immune system and 23 on experimental systems are particularly useful.

The term “inflammation” refers to both acute responses (i.e., responses in which the inflammatory processes are active) and chronic responses (i.e., responses marked by slow progression and formation of new connective tissue). Acute and chronic inflammation can be distinguished by the cell types involved. Acute inflammation often involves polymorphonuclear neutrophils; whereas chronic inflammation is normally characterized by a lymphohistiocytic and/or granulomatous response. Inflammation includes reactions of both the specific and non-specific defense systems. A specific defense system reaction is a specific immune system reaction response to an antigen (possibly including an autoantigen). A non-specific defense system reaction is an inflammatory response mediated by leukocytes incapable of immunological memory. Such cells include granulocytes, macrophages, neutrophils and eosinophils. Examples of specific types of inflammation are diffuse inflammation, focal inflammation, croupous inflammation, interstitial inflammation, obliterative inflammation, parenchymatous inflammation, reactive inflammation, specific inflammation, toxic inflammation and traumatic inflammation.

The term “leukocyte” means a white blood cell. Lymphocytes, monocytes and macrophages are examples of leukocytes.

The term “lymphocyte” refers to a mononuclear leukocyte that mediate humoral or cellular immunity.

The term “monocyte” refers to a mononuclear phagocytic leukocyte that circulates briefly in the bloodstream before migrating into the tissues where it becomes a macrophage.

The term “major histocompatibility complex” (MHC) refers to a plurality of cell surface proteins and glycoproteins that are present on the surfaces of mammalian cells and which mediate cell-cell interactions (e.g., tissue compatibility in organ and tissue transplantation) and the immune response in a mammal. The MHC is encoded in humans by the HLA complex, in dogs by the DLA complex and in mice by the H-2 complex. The primary immunological function of MHC molecules is to bind and “present” pieces of antigenic molecules on the surfaces of cells for recognition (binding) by the antigen-specific T cell receptors (TCRs) of lymphocytes. Differential structural properties of class I and class II MHC molecules account for their respective roles in activating different populations of T lymphocytes (T cells). Class I MHC molecules specifically bind CD8 molecules expressed on cytotoxic T lymphocytes (T_{C} cells). Class II MHC molecules specifically bind CD4 molecules expressed on helper T lymphocytes (T_{H} cells). Class I MHC expression is widespread on virtually every cell of the body. This is consistent with the protective function of T_{C} cells which continuously survey cell surfaces and kill cells harboring metabolically active microorganisms infecting these cells. Class II MHC expression is restricted to “antigen presenting cells” (or APCs). This is consistent with the functions of T_{H} cells which are locally activated wherever these cells encounter macrophages, dendritic cells, follicular dendritic cells or B lymphocytes (B cells) that have internalized and processed antigens produced by pathogenic organisms and display pieces of these antigens using class II MHC molecules.

The term “patient” includes human and other mammalian subjects that receive either prophylactic or therapeutic treatment.

The term “PBMC” or “peripheral blood mononuclear cell” refers to mononuclear cells circulating in the peripheral blood stream of a mammal.

The term “substantial identity” means that two peptide sequences, when optimally aligned, such as by the programs GAP or BESTFIT using default gap weights, share at least 85 percent sequence identity, preferably at least 80 or 90 percent sequence identity, more preferably at least 95 percent sequence identity or more (e.g., 99 percent sequence identity or higher). Preferably, residue positions which are not identical differ by conservative amino acid substitutions.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, (1981) Adv. Appl. Math. 2:482, by the homology alignment algorithm of Needleman & Wunsch, (1970) J. Mol. Biol. 48:443, by the search for similarity method of Pearson & Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra). One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (http://www.ncbi.nlm.nih.gov/). Typically, default program parameters can be used to perform the sequence comparison, although customized parameters can also be used. For amino acid sequences, the BLASTP program uses as defaults a wordlength (W) of 3, an expectation (E) of 10, and the BLOSUM62 scoring matrix. (See Henikoff & Henikoff, (1989) Proc. Natl. Acad. Sci. USA 89, 10915).

For purposes of classifying amino acids substitutions as conservative or nonconservative, amino acids are grouped as follows: Group I (hydrophobic side chains): norleucine, met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

The term “substantially pure” or “isolated” means an object species has been identified and separated and/or recovered from a component of its natural environment such as tissue culture cells or a natural source. For example, a substantially pure or isolated rhesus CMV IL-10 or human CMV IL-10 protein produced by recombinant means in a non-human cell is free of other viral proteins with which it exists in nature. Usually, the object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 to 90 percent by weight of all macromolecular species present in the composition and more preferably 90, 95, 99 or 99.9 percentage. Most preferably, the object species is purified to essential homogeneity (contaminant species cannot be detected in the composition by conventional detection methods) wherein the composition consists essentially of derivatives of a single macromolecular species.

The term “superantigen” means any substance that binds to the V_{β} domain of the T-cell receptor and resides in the chain of class II MHC molecules. It induces activation of all T cells that express T-cell receptors with a particular V_{β} domain. It functions as a potent T cell mitogen.

As used herein “T cell” refers to a lymphocyte that matures in the thymus and expresses a T-cell receptor, CD3 and CD4 or CD8. There are several recognized T-cell subpopulations.

As used herein “T_{DTH} cell” refers to a cell, generally a CD4⁺ lymphocyte, derived from a T_{H} cell that mediates delayed-type hypersensitivity.

As used herein “T_{H}1 response” refers to a response that produces a cytokine profile that supports inflammation and cell mediated responses. The “T_{H}1 subset” refers to a subset of T helper cells responsible for the T_{H}1 response.

II. General

The present invention provides pharmaceutical compositions and prophylactic and therapeutic methods of treatment for immune disorders using rhesus or human CMV IL-10. These methods are premised in part on data provided by the present application showing that contacting PBMCs with rhesus CMV IL-10 or human CMV IL-10 inhibits PBMC proliferation and cytokine production and treatment of monocytes with CMV IL-10 reduces cytokine production, monocyte surface expression of classical class I MHC and class II MHC molecules and increases monocyte surface expression of the nonclassical class I MHC molecule, HLA-G. Therefore rhesus CMV IL-10 and human CMV IL-10 can be used to inhibit lymphocyte proliferation and underlying cellular events both in vitro and in vivo. In both in vivo and in vitro methods, PBMCs are contacted with rhesus or human CMV IL-10 in a sufficient amount to inhibit PBMC proliferation. In vivo methods are useful for treating a number of immune disorders, such as cytokine mediated diseases, cell mediated cytotoxicity immune disorders, hypersensitivity immune disorders, chronic immune disorders, graft rejection, and cancer.

III. Sources of Rhesus CMV IL-10 and Human CMV IL-10

As used herein, “rhesus cytomegalovirus interleukin 10” or “rhesus CMV IL-10” is defined as a protein which has an amino acid having substantial identity to a known sequence of rhesus CMV IL-10 as described in Lockridge et al., Virology (2000) 268:272-280, which is incorporated herein by reference. For the purposes of this invention, some methods used glycosylated (e.g., produced in eukaryotic cells such as yeast or CHO cells) rhesus CMV IL-10 and some methods used unglycosylated (e.g., chemically synthesized or produced in prokaryotic cells, such as E. coli) rhesus CMV IL-10.

As used herein, “human cytomegalovirus interleukin 10” or “human CMV IL-10” is defined as a protein which has an amino acid having substantial identity to a known sequence of human CMV IL-10 as discussed in Kotenko et al., PNAS (2000) 97(4):1695-1700, which is incorporated herein by reference. For the purposes of this invention, some methods used glycosylated (e.g., produced in eukaryotic cells such as yeast or CHO cells) human CMV IL-10 and some methods used unglycosylated (e.g., chemically synthesized or produced in prokaryotic cells such as E. coli) human CMV IL-10

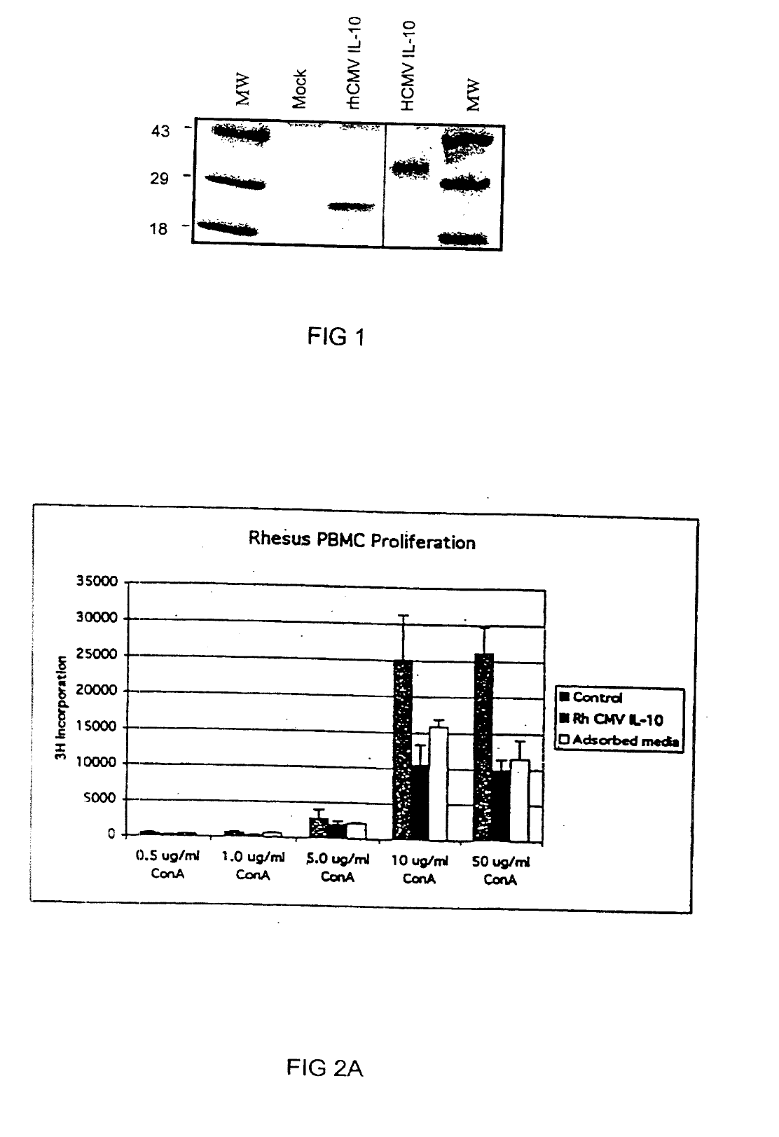

Rhesus CMV IL-10 or human CMV IL-10 suitable for use in the present invention can be obtained from a number of sources. For example, rhesus CMV IL-10 or human CMV IL-10 can be isolated from culture media of transfectants capable of secreting the proteins. (See FIG. 1). Additionally, rhesus CMV IL-10 or human CMV IL-10 or active fragments thereof can be chemically synthesized using standard techniques known in the art. See, e.g., Merrifield, (1986), Science 233:341-347 and Atherton et al., Solid Phase Peptide Synthesis, A Practical Approach, 1989, IRL Press, Oxford, which are incorporated herein by reference.

Rhesus CMV IL-10 or human CMV IL-10 can be obtained by recombinant techniques using isolated nucleic acids encoding the rhesus CMV IL-10 or the human CMV IL-10 polypeptide. General methods of molecular biology are described, e.g., by Sambrook et al., 1989, Molecular Cloning, A Laboratory Manual, 2d Ed., Cold Spring Harbor, N.Y. and Ausubel et al. (eds.). Current Protocols in Molecular Biology, Green/Wiley, New York (1987 and periodic supplements), which are incorporated herein by reference. The appropriate sequences can be obtained from cDNA libraries using standard techniques. DNA constructs encoding rhesus CMV IL-10 or human CMV IL-10 can also be prepared synthetically by established standard methods, e.g., in an automatic DNA synthesizer, and then purified, annealed, ligated and cloned in suitable vectors. See Atherton et al., Ibid., incorporated herein by reference. Polymerase chain reaction (PCR) techniques can be used. See, e.g., PCR Protocols: A Guide to Methods and Applications, 1990, Innis et al. (Ed.), Academic Press, New York, incorporated herein by reference. The DNA constructs can contain the entire native sequence of rhesus CMV IL-10 or human CMV IL-10 or sequence encoding a peptide having substantial identity to the entire native sequence or an active fragment thereof.

Peptides having substantial identity to rhesus CMV IL-10 or human CMV IL-10 can be prepared with various objectives in mind, including increasing serum half-life, facilitating purification or preparation, improving therapeutic efficacy, and lessening the severity or occurrence of side effects during therapeutic use. The peptides having substantial identity to rhesus CMV IL-10 or human CMV IL-10 are usually predetermined peptides not found in nature, although others can be post-translational variants, e.g., glycosylation variants or proteins which are conjugated to polyethylene glycol (PEG), etc. Such variants can be used in the present invention as long as they retain the biological activity of rhesus CMV IL-10 or human CMV IL-10.

The nucleotide sequences used to transfect the host cells can be modified to yield rhesus CMV IL-10 or human CMV IL-10 mutants and fragments with a variety of desired properties. Such modified rhesus CMV IL-10 or human CMV IL-10 can vary from the naturally-occurring sequence at the primary level, e.g., by amino acid insertions, substitutions, deletions and fusions. These modifications can be used in a number of combinations to produce the final modified rhesus CMV IL-10 or human CMV IL-10 peptide.

When rhesus CMV IL-10 or human CMV IL-10 of the present invention are expressed in soluble form, e.g., as a secreted product of transformed yeast, bacterial, or mammalian cells, they can be substantially purified according to standard procedures of the art, including steps of ammonium sulfate precipitation, ion exchange chromatography, gel filtration, electrophoresis, affinity chromatography, and/or the like. “Enzyme Purification and Related Techniques,” Methods in Enzymology, 22:233-577 (1977), and Scopes, R., Protein Purification: Principles and Practice (Springer-Verlag, New York, 1982), incorporated herein by reference, provide guidance in such purifications. Likewise, when rhesus CMV IL-10 or human CMV IL-10 of the present invention are expressed in insoluble form, e.g., as aggregates, inclusion bodies, or the like, they can be substantially purified by standard procedures in the art, including separating the inclusion bodies from disrupted host cells by centrifugation or sonification, solublizing the inclusion bodies with chaotropic and reducing agents, diluting the solubilized mixture, and lowering the concentration of chaotropic agent and reducing agent so that the polypeptide takes on a biologically active conformation. The latter procedures are disclosed in the following references: Winkler et al., Biochemistry, 25: 4041-4045 (1986); Winkler et al., Biotechnology, 3: 992-998 (1985); Koths et al., U.S. Pat. No. 4,569,790; and European Patent Nos. 215 625 and 212 960, which are incorporated by reference.

VI. Cellular Responses

Both in vitro and in vivo, contacting PBMCs with rhesus or human CMV IL-10 results in a number of cellular responses, including an inhibition of proliferation (if cells are already proliferating) or prevention of proliferation (if cells are not already proliferating but are in conditions promoting proliferation of the cells). Other cellular events are also affected by treatment with rhesus or human CMV IL-10, including inflammatory reduced cytokine production, reduced monocyte surface expression of classical class I and class II MHC molecules and increased monocyte surface expression of the nonclassical class I MHC molecule, HLA-G.

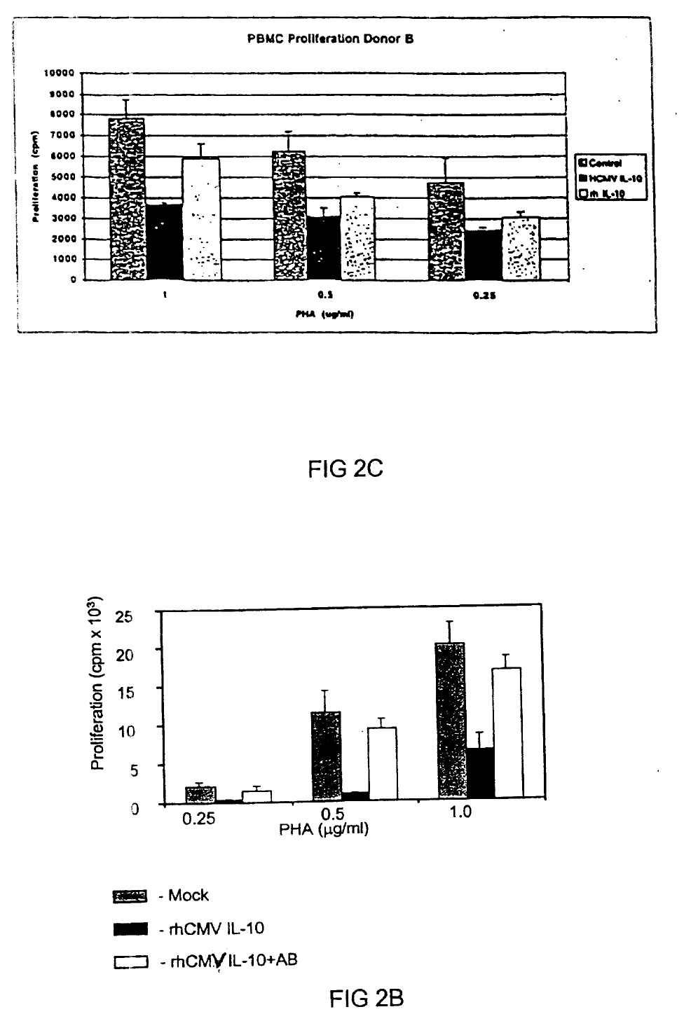

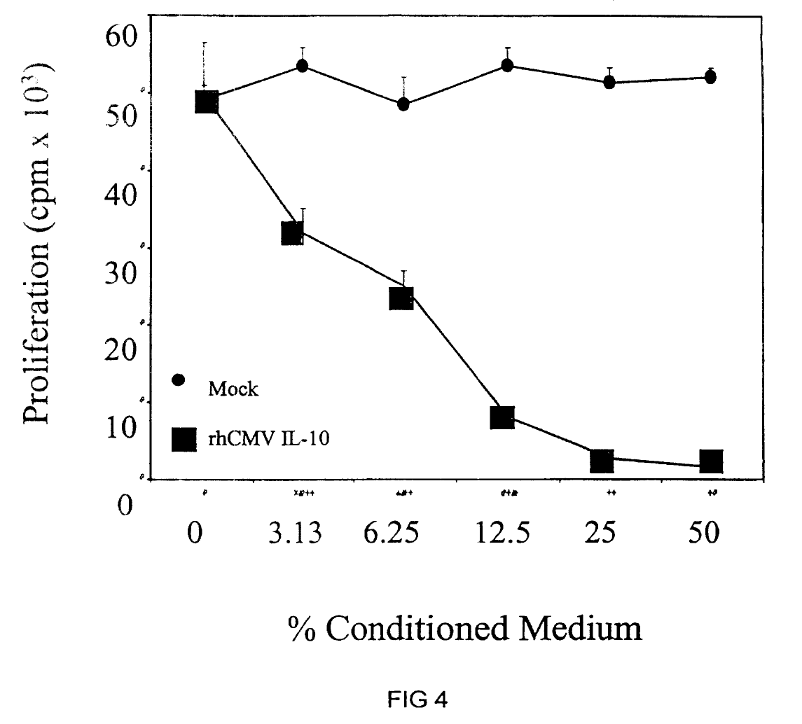

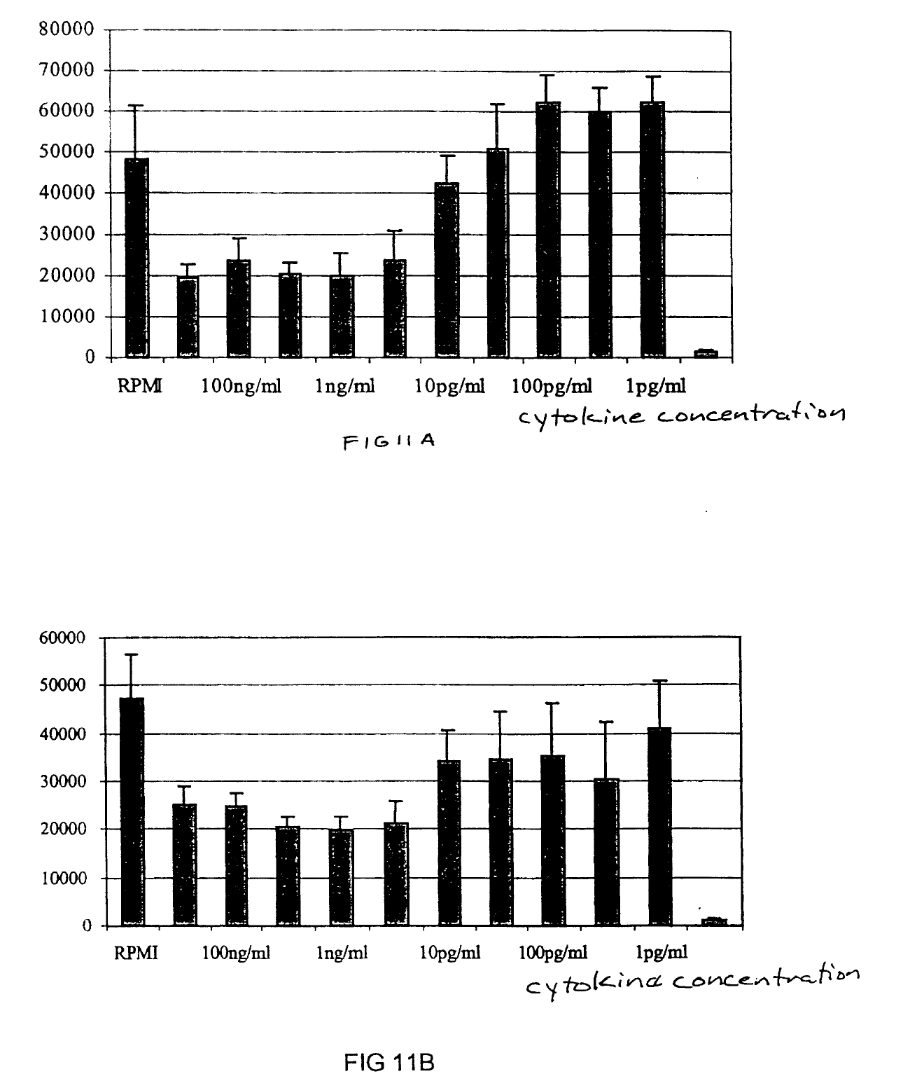

Rhesus and human CMV IL-10 detectably and significantly inhibit proliferation of PBMCs. In some methods, proliferation of PBMC populations contacted with rhesus or human CMV IL-10 is inhibited by at least 10, 25, 50, 75, 90 or 95%. In some methods, proliferation of PBMC populations contacted with rhesus or human CMV IL-10 is inhibited by 20-90% or 50-90%. Contact with rhesus CMV IL-10 or human CMV IL-10 inhibited proliferation of PHA stimulated human PBMCs in a dose dependent manner. In some methods, significant inhibition of the PBMC proliferative response was obtained when PBMCs were contacted with 100 ng/ml, 1 ng/ml, 10 pg/ml, 100 pg/ml, or 1 pg/ml of purified human CMV IL-10. In some methods, significant inhibition of the PBMC proliferative response was obtained when PBMCs were contacted with between about 100 ng/ml to 1 pg/ml of purified human CMV IL-10. (See Example 2, FIGS. 2A and 2B, and FIG. 4; Example 20, FIG. 2C; and, Example 21 and FIGS. 11A and 11B).

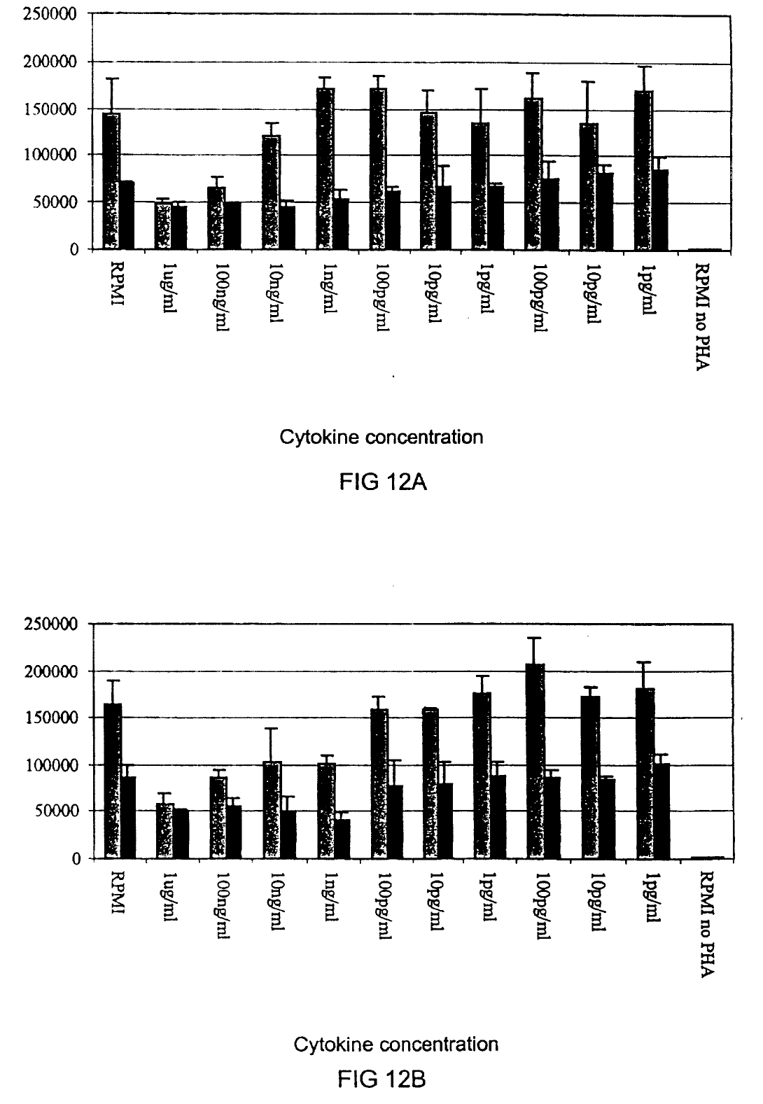

In vitro, proliferation can be assessed by measuring incorporation of labeled tritium as described in the Examples. A base line measurement can be made before contacting a population of PBMCs with rhesus or human CMV IL-10 and additional measurements can be made thereafter. Inhibition can be determined as a percentage of level of tritium incorporation before and after contacting PBMCs with rhesus or human CMV IL-10. Alternatively, a control reaction can be performed in parallel except that no CMV IL-10 is added. In this situation, percentage inhibition can be expressed as a percentage of the reaction mix in which CMV IL-10 is added relative to the control. Percentage inhibition of PBMCs in a patient can be performed by a similar process on a blood samples from the patient before and after treatment with that before treatment. Percentage inhibition can be expressed by comparing proliferation after treatment. Addition of a mouse monoclonal neutralizing antibody to the human IL-10 receptor to proliferating human PBMCs treated with human CMV IL-10 neutralized the activity of the human CMV IL-10. (See Example 22 and FIGS. 12A and 12B).

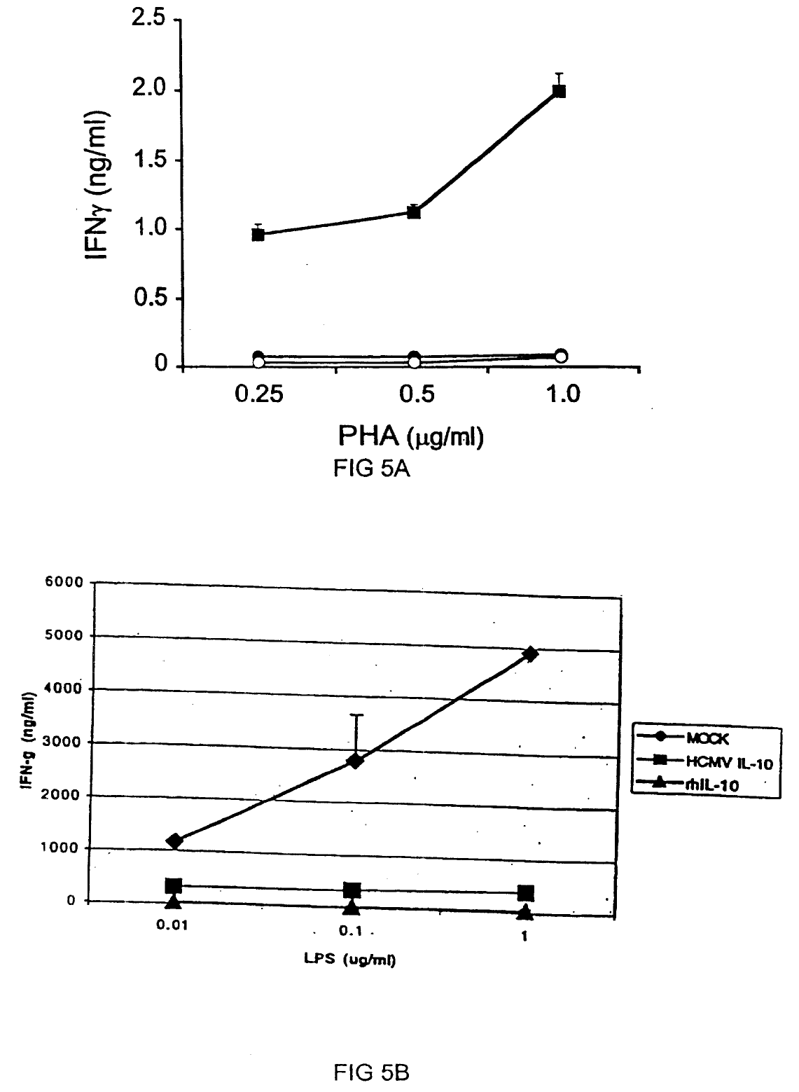

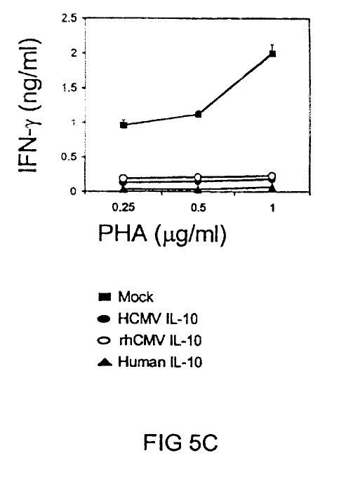

Similarly, treatment of PBMCs with rhesus or human CMV IL-10 detectably and significantly reduces levels of IFN-γ. (See Examples 4 and 5, and FIGS. 5A and 5C; and Examples 23 and 24, FIGS. 5B and 5C). In some methods, the reduction in IFN-γ levels in PBMC cultures contacted with rhesus CMV IL-10 or human CMV IL-10 is at least 10, 50, 75, 95, or 99%.

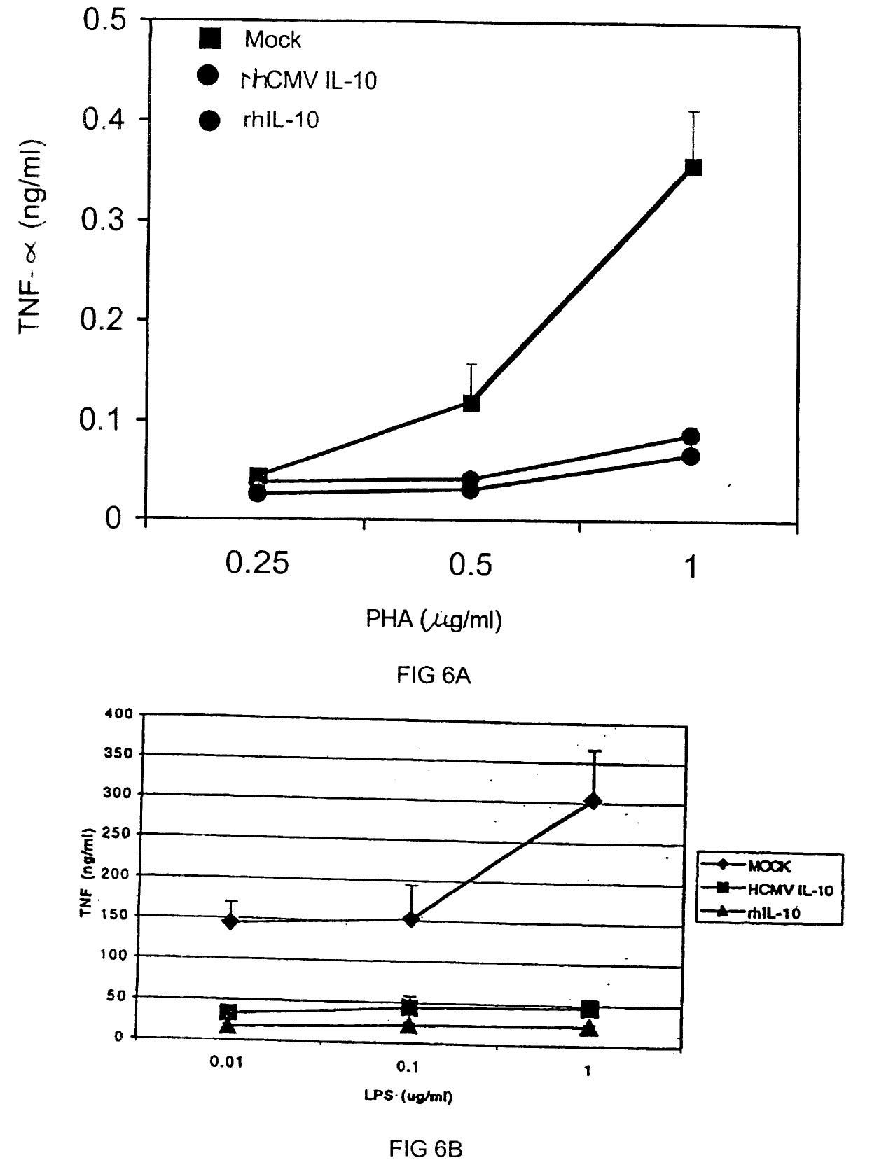

Treatment of PBMCs with rhesus or human CMV IL-10 detectably and significantly reduces levels of TNF-α. (See Examples 6 and 7, and FIG. 6A; and, Examples 25 and 26, FIG. 6B). In some methods, the reduction in TNF-α levels in PBMC cultures contacted with rhesus CMV IL-10 or human CMV IL-10 ranged is at least 10, 50, 75, 80, 90 or 95%.

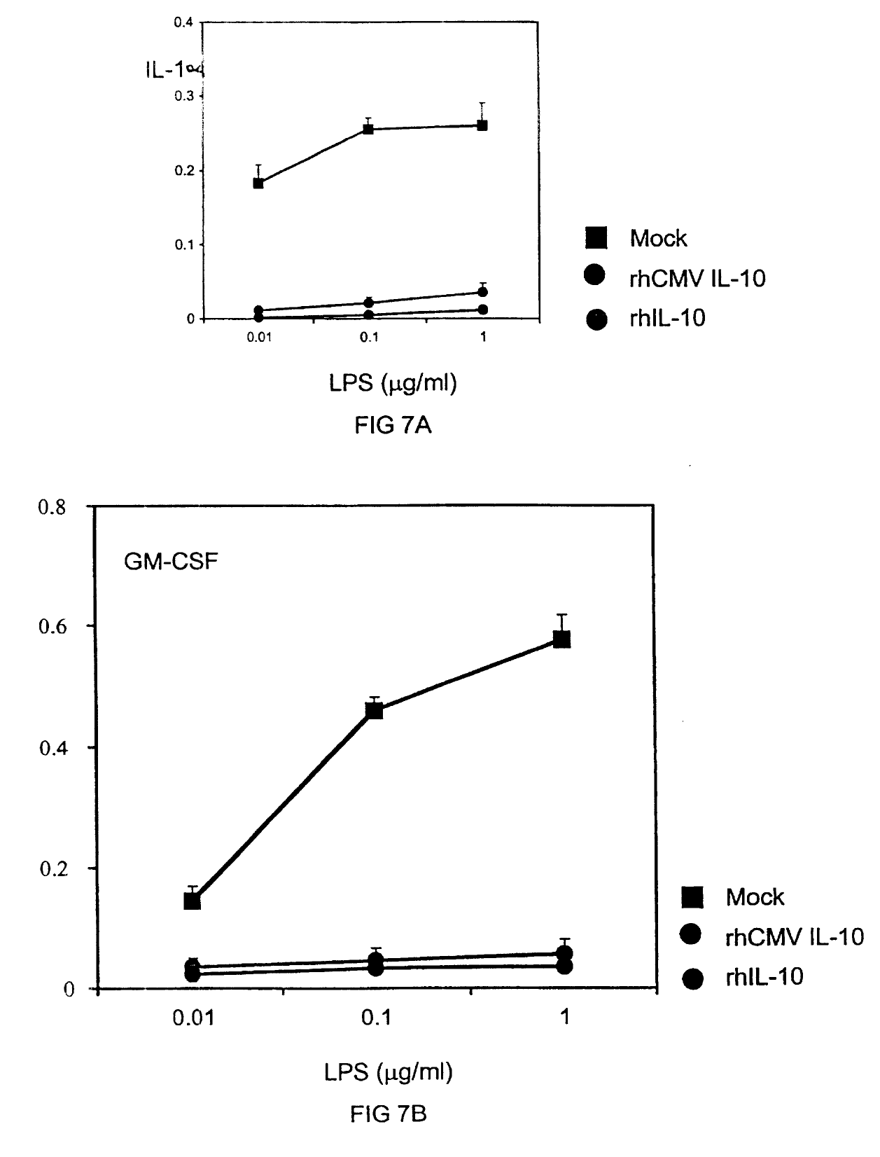

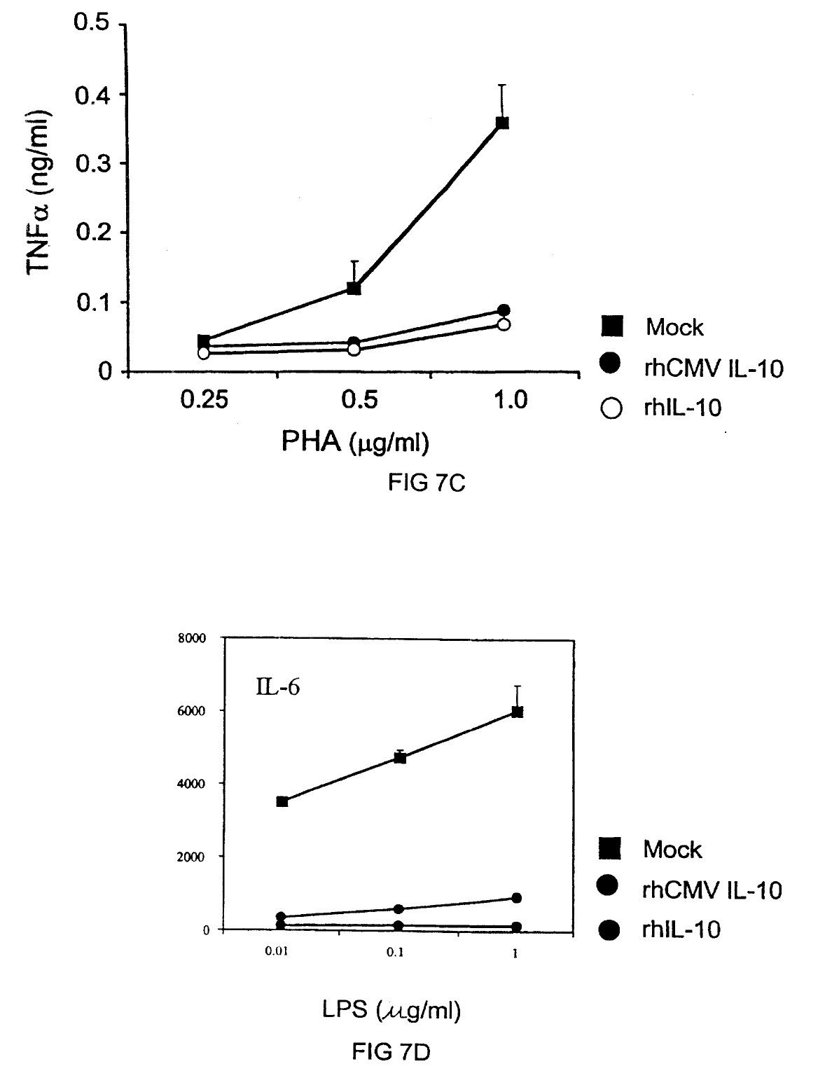

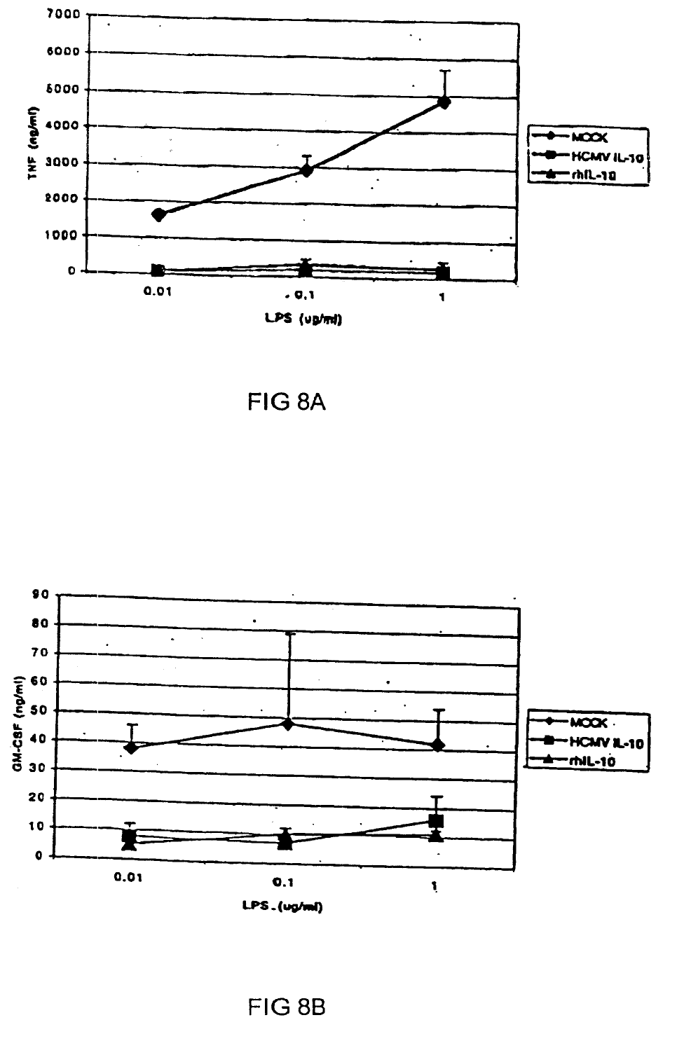

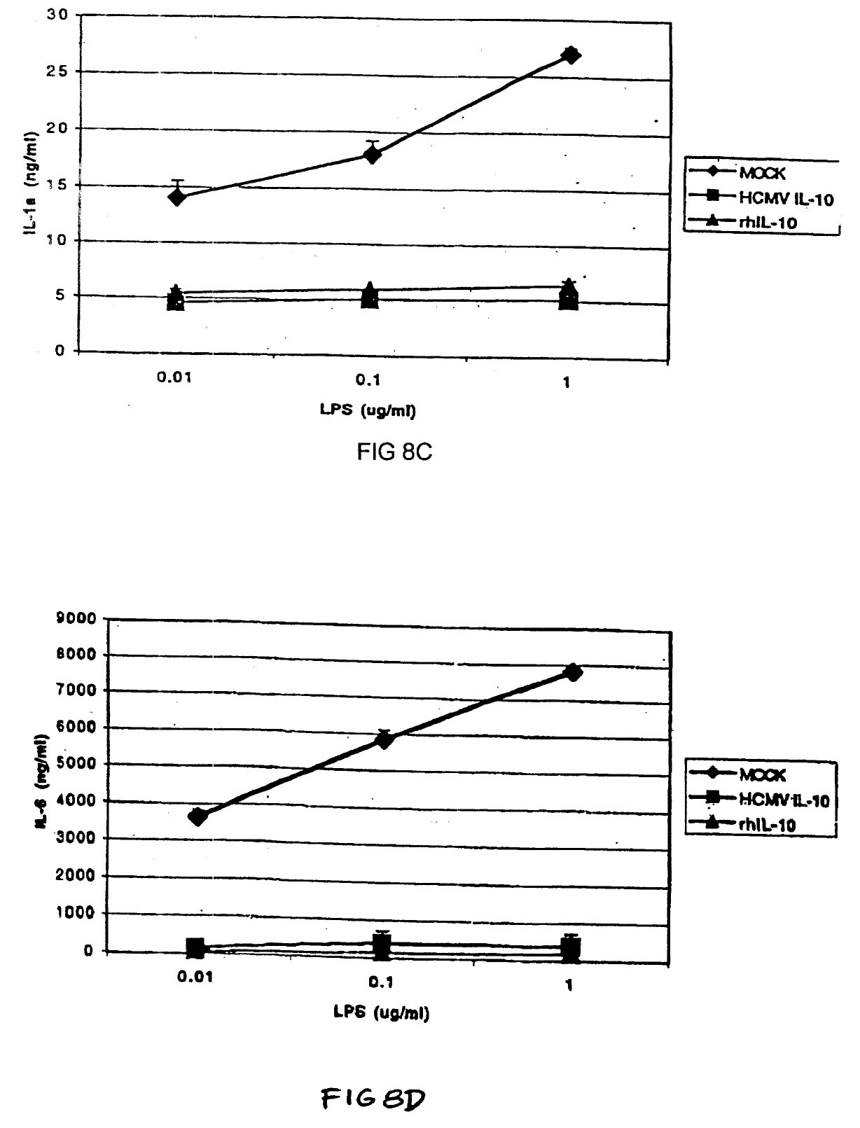

Human monocyte production of TNF-α, GM-CSF, IL-1α, and IL-6 are also significantly and detectably reduced in the presence of rhesus CMV IL-10. (See Examples 8 and 9, and FIG. 7C; Example 27 and FIG. 18; Examples 10 and 11, and FIG. 7B; Examples 12 and 13, and FIG. 7A; and, Example 14 and FIG. 7D, respectively). Human monocyte production of TNF-α, GM-CSF, IL-1α, and IL-6 are also significantly and detectably reduced in the presence of human CMV IL-10. (See Examples 27 and 28, FIG. 8A; Examples 29 and 30, and FIG. 8B; Examples 31 and 32, and FIG. 8C; Example 33 and FIG. 8D, respectively). In some methods, the reduction in TNF-α levels contacted with rhesus CMV IL-10 or human CMV IL-10 is at least 10, 25, 50, 75, 80 or 90%. Likewise, in some methods, the reduction in GM-CSF levels contacted with rhesus CMV IL-10 or human CMV IL-10 is at least 10, 25, 50, 75, 85 or 90%. In some methods, IL-1α levels are reduced by at least 10, 25, 50, 75, 85 or 90%.

Levels of the cytokines, TNF-α, IFN-γ, GM-CSF, IL-1α and IL-6 can be determined by ELISA, as described in the Example section, supra. A cytokine level is reduced when the cytokine level measured after a PBMC or monocyte population has been contacted with rhesus or human CMV IL-10 is lower than before contact with rhesus or human CMV IL-10.

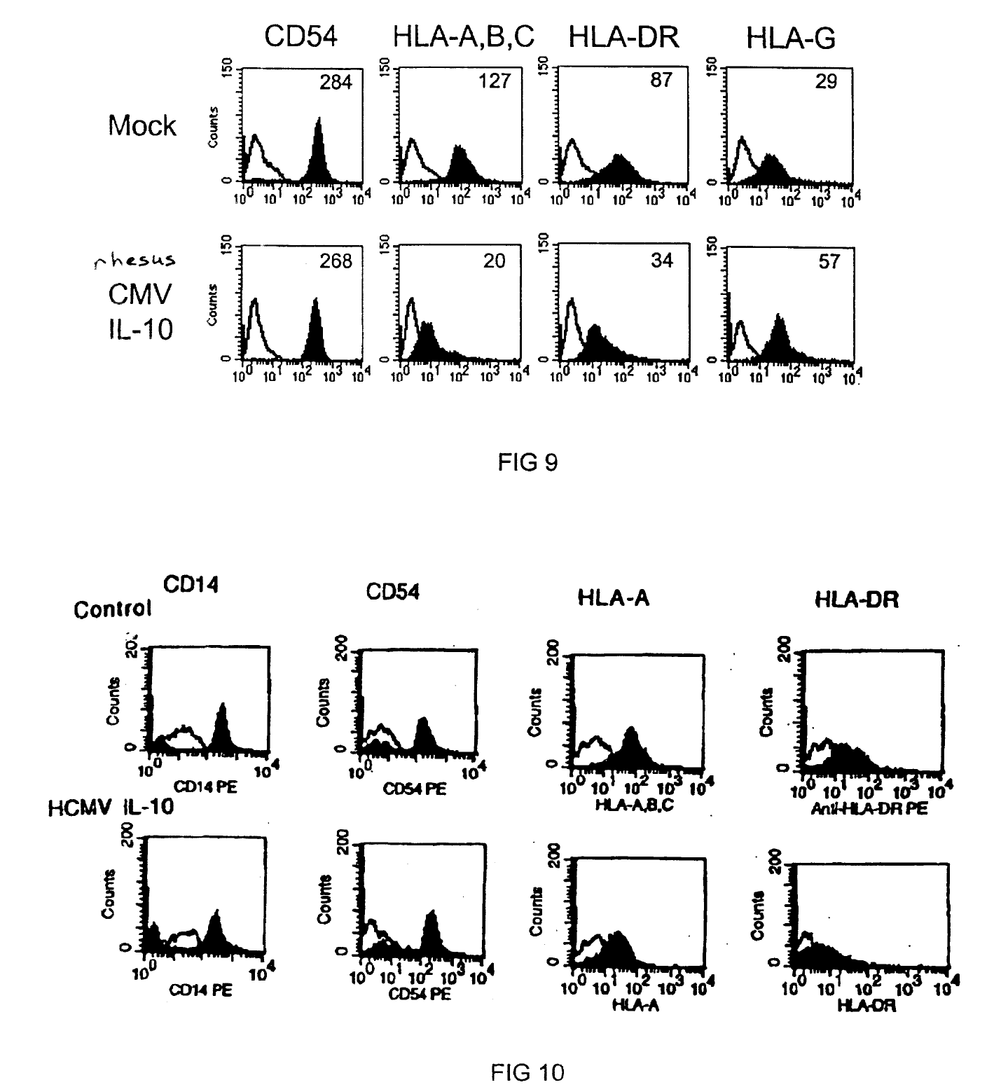

In some methods, human monocyte cell surface expression of classical class I MHC and class II MHC molecules are also reduced in the presence of rhesus CMV IL-10. (See Example 15 and FIG. 9, HLA-A, B, C panels; Example 16 and FIG. 9, HLA-DR panel). In some methods, human monocyte cell surface expression of classical class I MHC and class II MHC molecules are also reduced in the presence of human CMV IL-10. (See Example 34 and FIG. 10, HLA-A, B, C panels; Example 35 and FIG. 10, HLA-DR panel). In some methods, the reduction of cell surface expression of classical class I MHC molecules is at least 10, 20, 30 or 40%.

In some methods, human monocyte cell surface expression of nonclassical class I MHC molecules is increased in the presence of rhesus CMV IL-10. (See Example 17 and FIG. 9, HLA-G panel). In some methods, human monocyte cell surface expression of non-classical class I MHC molecules is increased in the presence of human CMV IL-10. (See Example 35 and FIG. 21, HLA-HLA-G panel). In some methods, the increase of cell surface expression of nonclassical class I MHC molecules is at least 10, 20, 30 or 40%.

Levels of cell surface expression of MHC molecules and CD54 molecules can be detected by surface staining and flow cytometry, as described in the Example section, supra. A reduction of cell surface expression of the MHC or CD54 molecule occurs when fewer of the cell surface molecules are measured after a monocyte population has been contacted with rhesus or human CMV IL-10 than before contact with rhesus or human CMV IL-10.

VI. In Vitro Uses

Rhesus and human CMV IL-10 can be used in a number of in vitro or ex vivo methods. In some methods, cellular responses to these agents are analyzed to provide information to optimize dosage regimes of these agents in vivo. In some, methods rhesus and human CMV IL-10 are used as positive controls to screen other drugs for effects on lymphocyte proliferation. If the positive control inhibits proliferation of the lymphocytes, whereas a candidate drug does not in a parallel reaction, then it can be concluded that the test drug is ineffective. In other methods, rhesus and human CMV IL-10 are used as research reagents to inhibit proliferation of cells and thereby analyze underlying cellular processes associated with cellular physiology. In other methods, proliferating PBMCs are obtained from a patient with an immune disorder. The lymphocytes are treated with rhesus CMV IL-10 or human CMV IL-10 ex vivo and then returned to the patients.

In some in vitro methods, a PBMC population is contacted with a purified preparation of rhesus or human CMV IL-10. In some methods, the PBMC population is contacted with an unpure preparation, such as protein containing supernatant from CMV-infected cells, or from cells in which rhesus or human CMV-IL-10 is recombinantly expressed. In some methods, a PBMC population is contacted with a nucleic acid encoding rhesus or human CMV IL-10, and the encoded protein is expressed using a transcription translation system.

V. Immune Disorders

Immune disorders preventable or treatable by methods of the invention include but are not limited to the following.

A. Cytokine-Mediated Diseases

Defects in the complex regulatory networks governing the overexpression of cytokines have been implicated in various cytokine-mediated diseases, including endotoxin-induced septic shock and endotoxin-induced toxic shock.

1. Endotoxin-Induced Septic Shock

In endotoxin-induced septic shock (also known as bacterial septic shock), shock (i.e., peripheral blood supply is inadequate to return sufficient blood flow to the heart for normal function, particularly the transport of oxygen to organs and tissues) apparently develops because the endotoxins of bacterial cell walls stimulate macrophages to overproduce IL-1 and TNF-α. It is the high levels of IL-1 and TNF-α that cause septic shock. Patients who die of meningitis have been found to have higher levels of TNF-α than patients who recovered.

Endotoxin-induced septic shock is often a fatal disease. The condition can develop within only a few hours of infection by some gram-negative bacteria, including E. coli, Klebsiella pneumoniae, Pseudomonas aeruginosa, Enterobacter aerogenes, and Neisseria meningitis. The symptoms of bacterial septic shock include paleness of the skin, weak pulse, dilated pupils, staring of the eyes, increased and shallow breathing rate, decreased blood pressure, fever, diarrhea and wide spread blood clotting in various organs. Thus, reducing the levels of IL-1 or TNF-α, or both IL-1 and TNF-α in a patient suffering from endotoxin-induced septic shock by administering an effective dosage of rhesus CMV IL-10 or human CMV IL-10 would be beneficial.

Septic shock can be induced in mice. In Examples 37 and 38, mice with septic shock are treated with rhesus CMV IL-1 or human CMV IL-10. In Example 39, mice with lethal septic shock are treated with rhesus CMV IL-10 or human CMV IL-10.

2. Endotoxin-Induced Toxic Shock

Endotoxin-induced toxic shock (also known as toxic shock syndrome) is a rare and sometimes fatal disease caused by the release of endotoxins that act as super antigens. A variety of organisms produce toxins that act as super antigens. Bacterial superantigens include several enterotoxins, exfoliating toxins, toxic-shock syndrome toxin (TSST1) from Staphlycoccus aureas; pyrogenic exotoxins from Streptococcus pyrogens and Mycoplasma arthritidis supernatant (MAS). These antigens activate a large number of T cells resulting in excessive production of cytokines. Toxic-shock syndrome toxin has been shown to induce extremely high levels of IL-1 and TNF. Thus, reducing the levels of IL-1 and TNF in a patient suffering from endotoxin-induced toxic shock by administering an effective dosage of rhesus CMV IL-10 or human CMV IL-10 would be beneficial.

Experiments similar to Examples 37 and 38 can be performed to test mice having endotoxin-induced toxic shock or a condition resembling endotoxin-induced toxic shock with rhesus CMV IL-10 or human CMV IL-10.

The symptoms of endotoxin-induced toxic shock include a fever of 102° or more, fainting, diffuse macular erythematous rash (red colored spotting on the skin, particularly the palms and soles) and involvement of three or more of the following organ systems: gastrointestinal (vomiting or diarrhea), muscular (severe muscle pain), mucus membranes, renal (kidneys), hepatic, hematologic or central nervous system.

B. Cell-Mediated Cytotoxicity Immune Disorders

Defects in the functioning of the cell-mediated immune response have been implicated in various cell-mediate cytotoxicity immune diseases, such as graft-verses host disease. Cytotoxic T lymphocytes (CTLs) are generated by the activation of T cytotoxic (T_{c}) cells. CTLs have lytic capability and are critical in the recognition and elimination of altered self-cells (e.g., virus-infected cells and tumors). Cytotoxic T lymphocytes (CTLs) are generally CD8⁺ are therefore class I MHC restricted. Since virtually all nucleated mammalian cells express class I MHC molecules, CTLs can recognize and eliminate almost any altered mammalian cell. This ability of CTLs to recognize and eliminate almost any altered mammalian cell can result in cell-mediated cytotoxicity related immune diseases. Consequently, a decrease in cell surface expression of class I MHC molecules is expected to ameliorate or prevent cell-mediated cytotoxicity related diseases. Thus, decreasing cell surface expression of class I MHC molecules in a patient suffering from a cell-mediated cytotoxicity immune disease by administering an effective dosage of rhesus CMV IL-10 or human CMV IL-10 would be beneficial.

1. Graft-Verses-Host Disease

Graft-verses-host disease (GVHD) occurs as a result of in vivo cell-mediated cytotoxicity. The disease develops when immunocompetent lymphocytes are introduced into an allogeneic recipient whose immune system is compromised. The grafted lymphocytes begin to attack the recipient and the recipient's compromised state prevents an immune response against the graft. The grafted lymphocytes are carried to the spleen, where they begin to proliferate in response to the allogenetic MHC antigens of the recipient. This proliferation induces an influx of recipient cells to the spleen and results in splenomegaly. The intensity of GVHD can be quantitated by calculating the spleen index (SI). A spleen index of 1.3 or greater is considered to be indicative of GVHD. Enlargement of the spleen is a result of proliferation of both CD4⁺ and CD8⁺ T-cell populations.

In Example 40, mice having GVHD are tested with rhesus CMV IL-10 or human CMV IL-10 and the SI of the mice is determined.

GVHD often develops in patients after transplantation of bone marrow into those patients who have leukemia or have received radiation exposure, immunodeficiency diseases or autoimmune anemias. The symptoms of GVHD include diarrhea, skin lesions, jaundice, spleen enlargement and death. Often, the epithelial cells of the skin and the gastrointestinal tract become necrotic causing the skin and intestinal lining to slough.

C. Hypersensitive Immune Disorders

Hypersensitivity reactions are inflammatory reactions with the humoral or cell-mediate branches of the immune system that lead to extensive tissue damage or even death. The reactions are classified into four main types, type I, type II, type III and type IV according to the mechanism that induces them. A type I response or IgE-mediated hypersensitivity is an immune disorder induced by allergens. The mediators of a type I reaction can be classified as primary or secondary. Cytokines, including TNF-α, are a secondary mediator of a type I hypersensitivity response and contribute to the clinical manifestations of type I hypersensitivity. A type IV or delayed-type hypersensitivity (DTH) reaction involves the cell-mediated branch of the immune system. Antigen activation of sensitized T_{DTH} cells induces the release of various cytokines that cause macrophages to accumulate and become activated. Antigen activation can be a result of intracellular pathogens or contact antigens. The cytokines released include IL-2, IL-3, IL-6, GM-CSF, IFN-γ, macrophage-inhibition factor (MIF), and TNF-β. The net effect of these cytokines is to cause an accumulation and activation of macrophages, which release lytic enzymes and cause tissue damage.

1. Type I Hypersensitivity Immune Disorders

a. Allergic Response

Many different allergens can produce and allergic response including, pollen, food, dust, fumes, insect products, viral antigens and bacterial antigens. The symptoms of an allergic reaction include bronchospasm, edema, mucous secretion and inflammation. In asthma, the allergic response develops in the lower respiratory tract. The symptoms of asthma include bronchoconstriction. Airway edema, mucus secretion and inflammation contribute to the bronchial constriction. Decreasing the symptoms of an allergic response in a patient suffering from such a response by administering rhesus CMV IL-10 or human CMV IL-10 would be beneficial. Type 1 hypersensitivity induced by allergic reactions can be modeled in rodents. In Example 41, rats with type 1 hypersensitivity are treated by administering rhesus CMV IL-10 or human CMV IL-10.

2. Delayed-Type Hypersensitivity Reactions Resulting in a Disease State

a. Intracellular Pathogens Inducing DTH

DTH plays an important role in host defense against intracellular pathogens. A variety of pathogens and contact antibodies can induce a DTH response. The initial immune response is nonspecific and often results in significant damage to healthy tissue. Although healthy tissue can be damaged, the patient can successfully eliminate cells infected by intracellular pathogens. When this defense process is not entirely effective, the continued presence of the pathogen's antigens can provoke a chronic DTH reaction. The chronic DTH reaction is characterized by excessive numbers of macrophages and the continued release of lytic enzymes resulting in tissue destruction. Thus, the DTH response to an intracellular pathogen can cause such extensive tissue damage that the DTH response is pathologic condition. The granulomatous skin lesion seen with Mycobacterium leprae and the lung cavitation seen with Mycobacterium tuberculosis infections are examples of such pathology resulting from a chronic DTH reaction. Chronic DTH responses can result in granulomatous disease.

There are many intracellular pathogens capable of inducing the tissue destruction characteristic of a DTH reaction. The following intracellular bacteria are capable of inducing a DTH reaction: Mycobacterium leprae, Mycobacterium tuberculosis, Listeria monocytogenes, and Brucella abortus. The following intracellular fungi are capable of inducing a DTH reaction: Pneumocystis carinii, Candida albicans, Histoplasma capsulatum, and Cryptococcus neoformans. The intracellular parasite Leishmania sp. is capable of inducing a DTH reaction. The following intracellular viruses are capable of inducing a DTH reaction: herpes simplex virus, variola, and measles. Thus, decreasing production of GM-CSF and IFN-γ in a patient experiencing pathological tissue destruction due to a DTH response to an intracellular parasite infection by administering an effective dosage of rhesus CMV IL-10 or human CMV IL-10 would be beneficial. A model of delayed hypersensitivity (type IV) reactions induced by intracellular pathogens can be modeled in rodents. In Example 42, mice with type 2 hypersensitivity are treated by administering rhesus CMV IL-10 or human CMV IL-10.

b. Contact Antigens Inducing DTH

Many contact-dermatitis reactions, including the responses to formaldehyde, trinitrophenol, nickel, turpentine, cosmetics, hair dyes, poison oak, and poison ivy are mediated by DTH responses. These substances form complexes with skin proteins. The complexes are then internalized by APCs in the skin (e.g., Langerhans cells), then processed and presented along with class II MHC molecules, resulting in activation of sensitized T_{DTH} cells. A subsequent exposure to the complex results in T_{DTH} cell activation and cytokine production. After the second exposure to the complex, the secreted cytokines cause macrophage accumulation at the exposure site. The activation of these macrophages and the release of their lytic enzymes result in the tissue damage characteristic of a contact-dermatitis reaction. Thus, decreasing cell surface expression of class I MHC molecules and production of GM-CSF and IFN-γ in a patient suffering from a contact-dermatitis by administering an effective dosage of rhesus CMV IL-10 or human CMV IL-10 would be beneficial.

D. Chronic Immune Disorders

Chronic inflammation develops because of the persistence of an antigen. Some microorganisms are resistant to phagocytosis. Such organisms often induce a chronic inflammatory response, resulting in significant tissue damage. Chronic inflammation also occurs in a number of autoimmune diseases in which self-antigens are continually activating T cells. IFN-γ and TNF-α play a central role in chronic inflammation. The accumulation and activation of macrophages is characteristic of chronic inflammation. Among other functions, activated macrophages have increased cytokine production and increased expression of class II MHC molecules. Thus, decreasing production of IFN-γ and TNF-α, and cell surface expression of class II MHC molecules and in a patient suffering from a chronic inflammatory response by administering an effective dosage of rhesus CMV IL-10 or human CMV IL-10 would be beneficial.

Chronic inflammatory diseases are often accompanied by cachexia, a condition having the symptoms of general ill health, malnutrition and wasting.

1. Granulomatis Disease

Cytokines released by the chronically activated macrophages also stimulate fibroblast and collagen production. A type of scar tissue develops at the site by a process called fibrosis. Generally, a wound healing reaction, fibrosis can interfere with normal tissue function. Chronic inflammation often leads to the formulation of a granuloma. Granulomatous diseases are characterized by granular tumors or growths, usually of lymphoid or epithelioid cells. Symptoms of granulomatous diseases include dermatitis, diarrhea, stomatitis, osteomyelitis, brain abscess, pulmonary disease and hepatosplenomegaly.

2. Chronic Inflammatory Diseases

Studies suggest that regions of plump endothelial cells resembling high-endothelial venules (HEVs) appear along the vasculature in tertiary extralymphoid sites of chronic inflammation. IFN-γ and TNF-α are suspected to play a role in the induction of HEV-like regions along the vasculature. (See Girard and Springer (1995) Immunol. Today 16:449, hereby incorporated by reference).

These HEV-like regions have been observed in a number of chronic inflammatory diseases, such as rheumatoid arthritis, Crohn's disease, ulcerative colitis, Graves' disease, Hashimoto's thyroiditis and insulin-dependent diabetes mellitus. (Rheumatoid arthritis, Graves' disease, Hashimoto's thyroiditis and insulin-dependent diabetes mellitus are also considered autoimmune diseases). Development of this HEV-like vasculature is thought to facilitate a large-scale influx of leukocytes, and thus, contribute to chronic inflammation. Thus, decreasing production of IFN-γ and TNF-α in a patient suffering from a chronic inflammatory response by administering an effective dosage of rhesus CMV IL-10 or human CMV IL-10 would be beneficial.

Some chronic inflammatory disorders are the result of an autoimmune disease. Autoimmune diseases occur as a result of a spontaneous or induced malfunction of the immune system of mammals in which the immune system fails to distinguish between foreign immunogenic substances within the mammal and/or autologous (“self”) substances and, as a result, treats autologous (“self”) tissues and substances as if they were foreign and mounts an immune response against them. Autoimmune disease is characterized by production of either antibodies that react with self tissue, and/or the activation of immune effector T cells that are autoreactive to endogenous self antigens. Rheumatoid arthritis, inflammatory bowel disease, Graves' disease, Hashimoto's thyroiditis, systemic lupus erythematosus, multiple sclerosis, scleroderma and insulin-dependent diabetes mellitus are considered to be autoimmune diseases.

The following immune disorders are a result of chronic inflammation.

Rheumatoid arthritis is a chronic systemic disease primarily of the joints. The symptoms of rheumatoid arthritis are marked by inflammatory changes in the synovial membranes and articular structures (joints) and by atrophy and rarefaction (bone density decreases) of the bones. In late stages of Rheumatoid arthritis, deformity and ankylosis (immobility of the joint) develop. A model of rheumatoid arthritis can be induced in mice or rats by administering type II collagen in complete Freund's adjuvant. In Example 43, mice with rheumatoid arthritis are treated by administering rhesus CMV IL-10 or human CMV IL-10.

Inflammatory bowel disease (“IBD”) is a term used for those inflammatory diseases of the bowel of unknown etiology. IBD includes Crohn's disease and ulcerative colitis.

Crohn's disease is a chronic granulomatous (small grain-like body or growth) inflammatory disease involving any part of the gastrointestinal tract from the mouth to anus; but commonly involving the ileum (lower three-fifths of the small intestines) with scarring and thickening of the bowel wall. The symptoms of Crohn's disease include the presence of chronic diarrhea, increased bowel sounds, cramping, possibly evidenced by weight loss and aversion to eating.

Ulcerative colitis is a chronic, recurrent ulceration in the colon (part of the large intestine that extends from the cecum to the rectum, i.e. not the entire large intestine), chiefly of the mucosa and submucosa. The symptoms of ulcerative colitis include cramping, abdominal pain, rectal bleeding and loose discharges of blood, pus and mucus, with little fecal particles. Complications of ulcerative colitis include hemorrhoids, abscesses, fistulas, (abnormal, tube-like passages from the colon), perforation of the colon and carcinomas. Several mouse models of inflammatory bowel diseases exist. There are mouse models for Crohn's disease, and mouse models for ulcerative colitis. In Example 44, mice with IBD were treated by administering rhesus CMV IL-10 or human CMV IL-10.

Graves' disease is characterized by thyrotoxicosis with diffuse goiter, exophthalmos or pretibial myxedema or any combination of the three. Symptoms of Grave's disease include fatigability, nervousness, emotional lability and irritability, heat intolerance and increased sweating, weight loss, palpitation, and tremor of the hands and tongue.

Hashimoto's thyroiditis is a progressive autoimmune disease of the thyroid gland, with lymphocyte infiltration of the gland and circulating antithyroid antibodies. Patients suffering from Hashimoto's thyroiditis have goiter and gradually develop hyperthyroidism.

Systemic lupus erythematous (SLE) is characterized by fever, weakness, arthritis, skin rashes, pleurisy and kidney dysfunction. Affected individuals produce autoantibodies to a vast array of tissue antigens, e.g., DBA, histones, red blood cells, platelets, leukocytes and clotting factors. Interactions of these autoantigens produces various symptoms. A mouse model of SLE occurs spontaneously in genetically susceptible N2B/WF₁ mice. In Example 45, mice with SLE are treated by administering rhesus CMV IL-10 or human CMV IL-10.

Multiple sclerosis (MS) is a disease where there are patches of demyelination throughout the white matter of the central nervous system, sometimes extending into the gray matter. Individuals having MS produce autoreactive T-cells that participate in the formation of inflammatory lesions along the myelin sheath of nerve fibers. The symptoms of multiple sclerosis include weakness, incoordination, speech disturbances, visual complaints and paresthesia (sensation of numbness, prickling or tingling).

Scleroderma is recognized as an autoimmune disease resulting in over-production of collagen in connective tissue. The most commonly identified symptom of scleroderma is a gradual tightening of the skin, usually in the extremities, such as the hands, feet and face. Affected individuals can develop CREST syndrome. CREST is an acronym for calcinosis, Reynaud's phenomenon, esophageal dysfunction, sclerodactyly and telangiectasia.

Diabetes mellitus is a general term of disorders characterized by excessive urine excretion. The symptoms of diabetes, including insulin-dependent diabetes, include excretion of large amounts of urine with a low specific gravity, dehydration, great thirst which is often accompanied by a voracious appetite, loss of strength and emaciation.

Uveitis is an inflammation of part of or all of the uvea, (the vascular middle coat of the eye, combining the iris, ciliary body and choroid) commonly involving the other tunics (the sclera and cornea, and the retina.). The symptoms of uveitis include swelling and irritation of the eye.

Hepatitis is an inflammation of the liver that can be caused by a variety of agents: viral infections, bacterial invasion and physical or chemical agents. Hepatitis A, B and C are the main types. The symptoms of hepatitis include fever, jaundice, an enlarged liver, nausea, vomiting, malaise, muscle and joint pain, photophobia (unusual intolerance of light) dark urine and clay colored stools.

Psoriasis is a common chronic, squamous dermatosis, marked by exacerbation and remissions and having a polygenic inheritance pattern. The symptoms of psoriasis are marked by the presence of rounded, dry scaling patches of various sizes, covered by a grayish white or silvery white scales that have a predilection for the extensor surfaces, nails, scalp, genitalia and the lumbosacral region.

E. Graft Rejection

Graft rejection is an immune disorder principally caused by cell-mediated response to alloantigens (primarily MHC molecules) expressed on cells of the graft. Both DTH and cell mediated cytotoxicity reactions (discussed supra) have been implicated in graft rejection. Commonly transplanted organs include, the cornea, lung, heart, liver, bone marrow, kidney, pancreas, blood and skin.

F. Cancer

Cytokines can be used to augment a patient's immune response to a cancer, such as leukemia. Mouse models of tumor growth can be established. SCID mice can be transplanted with primary human tumor cells. Normal mouse strains can be transplanted with a variety of well-characterized mouse tumor lines, including a mouse thymoma EL4 which has been transfected with OVA to allow easy evaluation of tumor specific antigen responses. In Example 46, mice with tumors are treated by administering rhesus CMV IL-10 or human CMV IL-10.

1. Leukemia

Cytokines can be used to augment a patient's immune response to a cancer, such as leukemia. Leukemia is a progressive, malignant disease of the blood forming organs, characterized by the distorted proliferation of and development of leukocytes (white blood cells) and their precursors in the blood and bone marrow. The symptoms of leukemia include anemia, infections, fatigue and bleeding from thrombocytopenia (abnormal decrease in the number of blood platelets), paleness, bone pain, liver and/or spleen enlargement, and purpura (bruising). Thus, treatment of a patient with leukemia by administering rhesus CMV IL-10 or CMV IL-10 to augment the patient's immune response to the cancer, and thereby reducing the symptoms of leukemia, would be beneficial.

VI. Therapeutic Methods, Pharmaceutical Compositions and Methods of Administration

A. Therapeutic Methods

In prophylactic application, pharmaceutical compositions or medicants are administered to a patient susceptible to, or otherwise at risk for developing an immune disorder in an amount sufficient to prevent, reduce, or arrest the development of an immune disorder. In therapeutic applications, compositions or medicants are administered to a patient suspected to develop, or already suffering from an immunological disease in an amount sufficient to reverse, arrest, or at least partially arrest, the symptoms of an immune disorder. In both prophylactic and therapeutic regimes, rhesus CMV IL-10 and human CMV IL-10 of the present invention are usually administered in several dosages until a sufficient response has been achieved. However, in both prophylactic and therapeutic regimes, rhesus CMV IL-10 and human CMV IL-10 of the present invention can administered in a single dosages until a sufficient response has been achieved. Typically, the treatment is monitored and repeated dosages can be given. Furthermore, the treatment regimes can employ similar dosages; routes of administration and frequency of administration to those used in treating other immune-mediated disorders.

The amount of rhesus CMV IL-10 or human CMV IL-10 protein and other active agents that can be combined with a carrier material to produce a single dosage form vary depending upon the disease treated, the mammalian species, and the particular mode of administration. The “effective dosage”, “pharmacologically acceptable dose” or “pharmacologically acceptable amount” for any particular patient can depend on a variety of factors including the activity of the specific compound employed, the species, age, body weight, general health, sex and diet of the patient being treated; the time and route of administration; the rate of metabolism or excretion; other drugs which are concurrently or have previously been administered; the type and severity of the immunological disease; severity of side-effects, whether the patient is animal or human, and the like. Usually the patient is human, but nonhuman mammals, including transgenic mammals, can also be treated. Full length or active fragments of rhesus CMV IL-10 may be administered in effective dosages; and, full length or active fragments of human CMV IL-10 may be administered in effective dosages.

For any rhesus CMV IL-10 or human CMV IL-10 reagent and other active agents used in the methods of the present invention, an effective dose for humans can be estimated initially from non-human animal models. An effective dose can be determined by a clinician using parameters known in the art. Generally, dosing begins with an amount somewhat less than the optimal effective dose. Dosing is then increased by small increments thereafter until an effective dosage is achieved. (See The Merck Manual of Diagnosis and Therapy, 16^{th} Edition, § 22, 1992, Berkow, Merck Research Laboratories, Rahway, N.J., which is incorporated herein by reference).

Dosages need to be titrated to optimize safety and efficacy. Toxicity and therapeutic efficacy of the compounds described herein can be determined by standard pharmaceutical procedures in experimental animals, e.g., by determining the LD₅₀, (the dose lethal to 50% of the population tested) and the ED₅₀ (the dose therapeutically effective in 50% of the population tested). The dose ratio between toxic and therapeutic effect is the therapeutic index and can be expressed as the ratio between LD₅₀ and ED₅₀. Compounds which exhibit high therapeutic indices are preferred. The data obtained from these nonhuman animal studies can be used in formulating a dosage range that is not toxic for use in humans. The dosage of such compounds lies preferably within a range of circulating concentrations that include the ED₅₀ with little or no toxicity. The exact formulation, route of administration and dosage can be chosen by the individual physician in view of the patient's condition. (See, e.g., Fingl et al. (1975) In: The Pharmacological Basis of Therapeutics, Chapter 1, which is incorporated herein by reference).

B. Pharmaceutical Compositions and Methods of Administration

Rhesus CMV IL-10 or human CMV IL-10 and other active agents can be delivered or administered to a mammal, e.g., a human patient or subject, alone, in the form of a pharmaceutically acceptable salt or hydrolyzable precursor thereof, or in the form of a pharmaceutical composition wherein the compound is mixed with suitable carriers or excipient(s) in an effective dosage. An effective regime means that a drug or combination of drugs is administered in sufficient amount and frequency and by an appropriate route to at least detectably prevent, delay, inhibit or reverse development of at least one symptom of an immunological disease. An “effective dosage”, “pharmacologically acceptable dose”, “pharmacologically acceptable amount” means that a sufficient amount of a rhesus CMV IL-10 or human CMV IL-10 or combination of rhesus CMV IL-10 or human CMV IL-10 with other active agents is present to achieve a desired result, e.g., preventing, delaying, inhibiting or reversing a symptom of an immune disorder or the progression of an immune disorder when administered in an appropriate regime.

Rhesus CMV IL-10 or human CMV IL-10 and other active agents that are used in the methods of the present invention can be administered as pharmaceutical compositions comprising the rhesus CMV IL-10 or human CMV IL-10, together with a variety of other pharmaceutically acceptable components. Pharmaceutical compositions can be in the form of solids (such as powders, granules, dragees, tablets or pills), semi-solids (such as gels, slurries, or ointments), liquids, or gases (such as aerosols or inhalants).

Suitable formulations for use in the present invention are found in Remington's Pharmaceutical Sciences (Mack Publishing Company 1985) Philadelphia, Pa., 17^{th} edition) and Langer, Science (1990) 249:1527-1533, which are incorporated herein by reference. The pharmaceutical compositions described herein can be manufactured in a conventional manner, i.e., mixing, dissolving, granulating, dragee-making, levigating, emulsifying, encapsulating, entrapping or lyophilizing processes.

Rhesus CMV IL-10 or human CMV IL-10 and other active agents can be formulated with common excipients, diluents or carriers, and compressed into tablets, or formulated as elixirs or solutions for convenient oral administration. Rhesus CMV IL-10 or human CMV IL-10 and other active agents can be can also be formulated as sustained release dosage forms and the like.

Administration of the compounds can be achieved in various ways, including oral, buccal, rectal, parenteral, intraperitoneal, intradermal, transdermal, intratracheal, intravenous, and intramuscular administration. The compound can be administered in a local rather than systemic manner, in a depot or sustained release formulation. In addition, the compounds can be administered in a liposome. Moreover, the compound can be administered by gene therapy.

For buccal administration, the compositions can take the form of tablets or lozenges formulated in a conventional manner.

For administration by inhalation, the compounds for use according to the present invention are conveniently delivered in the form of an aerosol spray preparation from pressurized packs, a nebulizer or a syringe sprayer, with the use of a suitable propellant, e.g., dichlorodifluoromethane, trichlorofluoromethane, dichlorotetrafluoroethane, carbon dioxide or other suitable gas, or from propellant-free, dry-powder inhalers. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compounds can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions or emulsions in oil-based or aqueous vehicles, and can contain formulator agents such as suspending, stabilizing and/or dispersing agents. The compositions are formulated as sterile, substantially isotonic and in full compliance with all Good Manufacturing Practice (GMP) regulations of the U.S. Food and Drug Administration.

Rhesus CMV IL-10 or human CMV IL-10 and other active agents can also be formulated in rectal compositions such as suppositories or retention enemas, e.g., containing conventional suppository bases such as cocoa butter, carbowaxes, polyethylene glycols or other glycerides, all of which melt at body temperature, yet are solidified at room temperature.

In addition to the formulations described previously, the compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, for example, as a sparingly soluble salt. (See, e.g., Urquhart et al., (1984), Ann Rev. Pharmacol. Toxicol. 24:199; Lewis, ed., 1981, Controlled Release of Pesticides and Pharmaceuticals, Plenum Press, New York, N.Y., U.S. Pat. Nos. 3,773,919, and 3,270,960, which are incorporated herein by reference).

Alternatively, other delivery systems for hydrophobic pharmaceutical compounds can be employed. Liposomes and emulsions are well known examples of delivery vehicles or carriers for hydrophobic drugs. In some methods, long-circulating, i.e., stealth, liposomes can be employed. Such liposomes are generally described in Woodle, et al., U.S. Pat. No. 5,013,556, the teaching of which is hereby incorporated by reference. The compounds of the present invention can also be administered by controlled release means and/or delivery devices such as those described in U.S. Pat. Nos. 3,845,770; 3,916,899; 3,536,809; 3,598,123; and 4,008,719; the disclosures of which are hereby incorporated by reference.

For administration by gene therapy, genetic material (e.g., DNA or RNA) of interest is transferred into a host to treat or prevent an immune related disease. In the present invention, the genetic material of interest encodes a for rhesus or human CMV IL-10 or a fragment thereof. According to one aspect of the invention, the genetic material should be therapeutically effective. Many such proteins, vectors, DNA are known per se. (See Culver, K. W., “Gene Therapy”, 1994, p. xii, Mary Ann Liebert, Inc., Publishers, New York, N.Y., incorporated herein by reference in its entirety). For the purposes of example only, vectors can be selected from the group consisting of Moloney murine leukemia virus vectors, adenovirus vectors with tissue specific promoters, herpes simplex vectors, vaccinia vectors, artificial chromosomes, receptor mediated gene delivery, and mixtures of the above vectors. Gene therapy vectors are commercially available from different laboratories such as Chiron, Inc., Emeryville, Calif.; Genetic Therapy, Inc., Gaithersburg, Md.; Genzyme, Cambridge, Mass.; Somtax, Alameda, Calif.; Targeted Genetics, Seattle, Wash.; Viagene and Vical, San Diego, Calif.

Adenoviruses are promising gene therapy vectors for genetic material encoding a for rhesus or human CMV IL-10 or a fragment thereof. Adenovirus can be manipulated such that it encodes and expresses the desired gene product (e.g., rhesus or human CMV IL-10 or a fragment thereof) and at the same time is inactivated in terms of its ability to replicate in a normal lytic viral life cycle. Adenovirus expression is achieved without integration of the viral DNA into the host cell chromosome, thereby alleviating concerns about insertional mutagenesis. Furthermore, adenoviruses have been used as live enteric vaccines for many years with an excellent safety profile (Schwartz, A. R. et al. (1974) Am. Rev. Respir. Dis. 109:233-238). Finally, adenovirus mediated gene transfer has been demonstrated in a number of instances including transfer of alpha-1-antitrypsin and CFTR to the lungs of cotton rats (Rosenfeld, M. A. et al. (1991) Science 252:431-434; Rosenfeld et al., (1992) Cell 68:143-155). Furthermore, extensive studies to attempt to establish adenovirus as a causative agent in human cancer were uniformly negative (Green, M. et al. (1979) PNAS USA 76:6606).

The pharmaceutical compositions also can comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Pharmaceutical compositions suitable for use in the present invention include compositions wherein the active ingredients are contained in an effective dosage.

VII. EXAMPLES

The following examples are provided by way of illustration, and not by way of limitation. Thus, selection of vectors and hosts, as well as concentration of reagents, temperatures, and other variable parameters are used to exemplify application of the present invention and are not to be considered as limitations thereof. Those of skill in the art will readily recognize non-critical parameters which can be varied to accomplish the invention described herein.

General methods of preparation, culture and purification of cells, PBMC proliferation assays, ELISA, cell surface staining and flow cytometry were carried out as follows.

PBMC Preparation, Culture and Purification

PBMCs were obtained from the peripheral blood of healthy rhesus macaques by hypotonic lysis of red blood cells or from the buffy coats of healthy human donors by Ficoll density gradient centrifugation. Rhesus macaque or human PBMCs were maintained in RPMI (Hyclone) plus 10% fetal calf serum (FCS, Hyclone) and 10 ng/ml recombinant human IL-2 (R&D Systems). Monocytes were purified from rhesus macaque or human PBMCs, prepared as described above, with anti-CD14 microbeads (Miltenyi) and cultured in RPMI plus 10% FCS.

PBMC Proliferation Assay

PBMC proliferation was measured by [3H]TdR incorporation assays which indicate changes in the rate of DNA replication. Human or rhesus macaque PBMCs were plated in 96-well culture dishes at 1×10⁵ cells per well and stimulated with PHA (Sigma) or Con A (Sigma) in the presence of 5-50% (v/v) supernatants from HEK-293 cell (ATTC #CRL-1573) transfectants expressing myc/his tagged rhesus CMV IL-10 or the control vector (mock). Rhesus CMV IL-10 was immunoprecipitated by incubation with 10 μg/ml anti-poly His antibody and protein A sepharose beads for 14 hours at 4° C. Purified recombinant hIL-10 was from R&D Systems. Plates were incubated at 37° C. for 72 hours, and DNA replication was determined by [3H]TdR incorporation (0.25 μCi/well, Amersham) during the last 18 hours of culture.

ELISA

Cytokine production by PBMCs and monocytes was measured by ELISA. Human or rhesus macaque PBMCs or monocytes were plated in 96-well culture dishes at 1×10⁵ cells per well and stimulated with PHA or LPS (Sigma) in the presence of mock or rhesus CMV IL-10 conditioned medium. After 48 hours, supernatants were harvested and cytokine production measured by ELISA. The 96-microtiter plates (Nalge Nunc) were coated with 4 μg/ml anti-cytokine capture antibody (anti-IFN-γ, anti-TNF-α, anti-GM-CSF, or anti-IL-1a respectively, from R&D Systems) for 16 hours at 4° C. After blocking with 1% BSA (in PBS), 100 μl of culture supernatant was added. After washing, biotinylated anti-cytokine antibodies (2 μg/ml, R&D Systems) were added, followed by streptavidin-HRP (Gibco BRL). Cytokine detection was via Genzyme Color Reagents (Genzyme Diagnostics). The reaction was terminated with 50 μl/well 2N HCl and samples were read at 450 nm in a microplate reader (Molecular Devices). Cytokine levels were determined by linear regression analysis using a standard curve.

Cell Surface Staining and Flow Cytometry

Monocyte cell surface expression of classical class I MHC and class II MHC molecules, HLA-G and CD54 molecules was detected by cell surface staining and flow cytometry. Rhesus macaque or human monocytes, obtained as described above, were stained with phycoerythrin conjugated antibodies to HLA-A, B, C (IgG₁), HLA-DR (IgG₂ₐ), CD54 (IgG₁) or the appropriate isotype control (Pharmingen) and analyzed using a FACS Scan and CellQuest software (Becton Dickinson). Staining with antibody 87G directed against HLA-G (see Lee, N. et al. (1995) Immunity 3:591-600) was followed by incubation with goat anti-mouse-PE secondary antibody. Antibody 87G was provided by Dr. D. E. Geraghty of the Fred Hutchinson Cancer Research Center, Seattle, Wash.

### Example 1

### Expression of Recombinant Rhesus CMV IL-10 in Human Embryonic Kidney Cells and Rhesus Cells

Human embryonic kidney (HEK) 293 cells were grown in DMEM (Hyclone) plus 5% FCS. The HEK293 cells were transfected with an expression vector encoding a C-terminal myc/His epitope-tagged rhesus CMV IL-10 protein from rhesus CMV strain 68.1. Supernatants were collected after 48 hours, clarified by low speed centrifugation and rhesus CMV IL-10 expression was verified by Western blot with an anti-poly-His monoclonal antibody (Invitrogen). Rhesus CMV IL-1 was detected using TMB Membrane Peroxidase Substrate (Kirkegaard & Perry Laboratories). Transfectant supernatants were harvested and found to express a protein of 26 kD, the predicted size of the epitope-tagged CMV IL-10 (see Lockridge et al. (2000) Virology 286:272-80) until at least 96 hours post-transfection. Mock conditioned medium was obtained from cells transfected with the control vector (pcDNA3.1-m/H, Invitrogen). Recombinant Rhesus CMV IL-10 can also be expressed in rhesus cells. (See FIG. 1).

### Example 2

### Rhesus CMV IL-10 Inhibits Proliferation of Rhesus and Human PBMCs

Recombinant rhesus CMV IL-10 was generated as described above. The supernatants, obtained as described above, were used to assay the effects of rhesus CMV IL-10 on rhesus macaque and human PBMC proliferation in the absence of other virally produced factors. PBMC proliferation was measured as described above. PBMCs from healthy rhesus macaques (n=5) and healthy human donors (n=9) were stimulated with PHA for 72 hours in the presence of rhesus CMV IL-10 or mock supernatants.

Every donor, regardless of species, showed marked inhibition of PBMC proliferation in the presence of rhesus CMV IL-10. PHA stimulated human PBMCs exhibited robust proliferation, but this proliferation was inhibited in the presence of rhesus CMV IL-10. (See FIG. 2B). While conditioned media constituted 50% of the total culture volume in these experiments, inhibition of proliferation was observed in cultures containing as little as 5% rhesus CMV IL-10 conditioned medium. Proliferation of human PBMCs cultured in the presence of the mock conditioned medium was not found to differ from human PBMCs cultured in fresh RPMI. Inhibition of PBMC proliferation in the presence of rhesus CMV IL-10 was directly dose dependent. (See FIG. 4).

To confirm that the inhibitory activity was solely a province of rhesus CMV IL-10, the human PBMC culture supernatants were depleted of rhesus CMV IL-10 by immunoprecipitation from the conditioned medium with an anti-poly His antibody (rhesus CMV IL-10+Ab). This resulted in partial restoration of rhesus PBMC proliferation (see FIG. 2A) and a near total restoration of human PBMC proliferation to levels observed with mock medium (see FIG. 2B).

### Example 3

### Rhesus CMV IL-10 More Consistently Inhibited Proliferation of PHA-Stimulated Human PBMCs than did Recombinant rhIL-10; Human CMV IL-10 More Consistently Inhibited Proliferation of PHA-Stimulated Human PBMCs than did Recombinant hIL-10

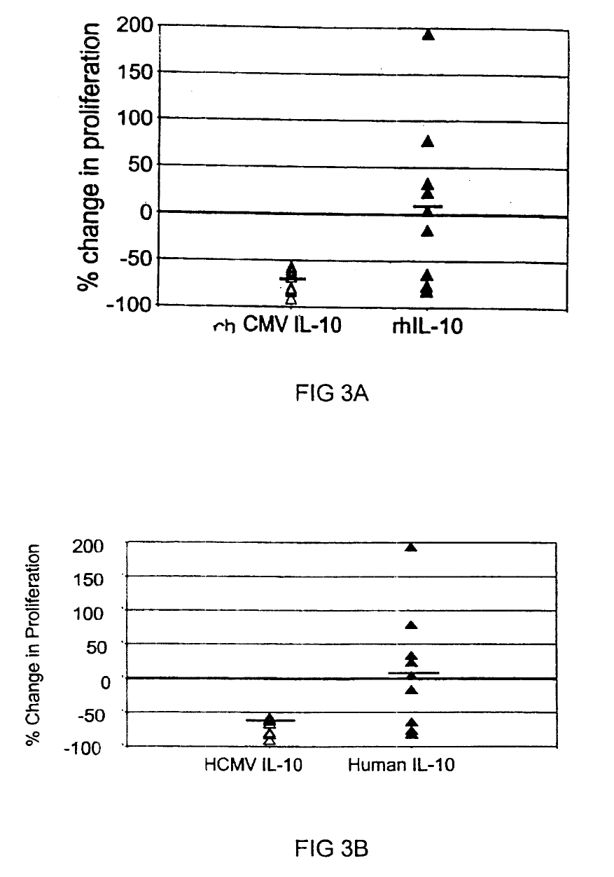

PBMCs from nine human donors were tested for effects on proliferation in the presence of rhesus CMV IL-10, 1 μg/ml recombinant hIL-10 conditioned media or mock conditioned media. PBMC proliferation was measured as described above. Rhesus CMV IL-10 inhibited human PBMC proliferation for all donors tested (57-91% inhibition). Extensive variability of human PBMC proliferation was observed with rhIL-10 treatment. Human PBMC proliferation for four donors was inhibited in the presence of 1 μg/ml hIL-10 (16-81% inhibition), while four other donors showed enhanced proliferation (increases of 24-194%) and one donor showed modest enhancement. (See FIG. 3A). Similarly proliferation of rhesus PBMCs were varied in response to rhIL-10. Results in FIG. 3A are expressed as percent increase in proliferation relative to control cultures from the same donor. Bar indicates mean change in proliferation for all donors.

PBMCs from nine human donors were tested for effects on proliferation in the presence of human CMV IL-10, 1 μg/ml recombinant hIL-10 conditioned media or mock conditioned media. PBMC proliferation was measured as described above. Human CMV IL-10 inhibited human PBMC proliferation for all donors tested. Extensive variability of human PBMC proliferation was observed with hIL-10 treatment. (See FIG. 3B). Similarly proliferation of rhesus PBMCs were varied in response to hIL-10. Results in FIG. 3B are expressed as percent increase in proliferation relative to control cultures from the same donor. Bar indicates mean change in proliferation for all donors.

### Example 4

### Rhesus CMV IL-10 Inhibits IFN-γ Synthesis by Human PBMCs

PHA-stimulated human PBMCs were cultured in the presence of mock or rhesus CMV IL-10 conditioned medium. After 48 hours, supernatant IFN-γ levels were assayed by ELISA as described above. While control human PBMC cultures produced IFN-γ in response to PHA stimulation, the levels of IFN-γ were decreased in cultures containing rhesus CMV IL-10. Production of IFN-γ was almost completely abrogated in the presence of rhesus CMV IL-10. (See FIGS. 5A and 5C).

### Example 5

### Effect of Rhesus CMV IL-10 on IFN-γ Synthesis by Human PBMCs is Comparable to the Effect of Recombinant rhIL-10 on IFN-γ Synthesis by Human PBMCs

PHA-stimulated human PBMCs were cultured in the presence of mock or 1 μg/ml recombinant rhIL-10 conditioned medium. After 48 hours, supernatant IFN-γ levels were assayed by ELISA as described above. While control human PBMC cultures produced IFN-γ in response to PHA stimulation, the levels of IFN-γ were decreased in cultures containing rhIL-10. The decrease in IFN-γ, levels in cultures containing rhIL-10 were comparable to the decrease in IFN-γ levels in cultures containing rhesus CMV IL-10. (See FIGS. 5A and 5C).

### Example 6

### Rhesus CMV IL-10 Inhibits TNF-α Synthesis by Human PBMCs

PHA-stimulated human PBMCs were cultured in the presence of mock or rhesus CMV IL-10 conditioned medium. After 48 hours, supernatant TNF-α levels were assayed by ELISA as described above. While control human PBMC cultures produced TNF-α in response to PHA stimulation, the levels of TNF-α were decreased in cultures containing rhesus CMV IL-10. Production of TNF-α was reduced by 81% to less than 50 pg/ml by rhesus CMV IL-10 treatment. (See FIG. 6A).

### Example 7

### Effect of Rhesus CMV IL-10 on TNF-α Synthesis by Human PBMCs is Comparable to the Effect of Recombinant hIL-10 on TNF-α Synthesis by Human PBMCs

PHA-stimulated human PBMCs were cultured in the presence of mock or 1 μg/ml recombinant hIL-10 conditioned medium. After 48 hours, supernatant TNF-α levels were assayed by ELISA as described above. While control human PBMC cultures produced TNF-α in response to PHA stimulation, the levels of TNF-α were decreased in cultures containing hIL-10. The decrease in TNF-α levels in cultures containing hIL-10 were comparable to the decrease in TNF-α levels in cultures containing rhesus CMV IL-10. (See FIG. 6A).

### Example 8

### Rhesus CMV IL-10 Inhibits TNF-α Synthesis by Human Monocytes

Primary monocytes were purified from human PBMCs as described above. Flow cytometric analysis was performed as described above and revealed the resulting cell population to be >99% CD14⁺. Human monocytes were stimulated with LPS in the presence of mock or rhesus CMV IL-10 conditioned medium. After 48 hours, supernatants were assayed for TNF-α by ELISA, as described above. In LPS-stimulated monocyte cultures, robust production of TNF-α was observed. In the presence of rhesus CMV IL-10 the level of TNF-α was greatly reduced. (See FIG. 7C).

### Example 9

### Effect of Rhesus CMV IL-10 TNF-α Synthesis by Human PBMCs is Comparable to the Effect of Recombinant hIL-10 on TNF-α Synthesis by Human Monocytes

Primary monocytes were purified from human PBMCs as described above. Flow cytometric analysis was performed as described above and revealed the resulting cell population to be >99% CD14⁺. Human monocytes were stimulated with LPS in the presence of mock or recombinant hIL-10 conditioned medium. After 48 hours, supernatants were assayed for TNF-α by ELISA as described above. In LPS-stimulated monocyte cultures, robust production of TNF-α was observed. The decrease in TNF-α levels in cultures containing hIL-10 were comparable to the decrease in TNF-α levels in cultures containing rhesus CMV IL-10. (See FIG. 7C).

### Example 10

### Rhesus CMV IL-10 Inhibits GM-CSF Synthesis by Human Monocytes

Primary monocytes were purified from human PBMCs as described above. Flow cytometric analysis was performed as described above and revealed the resulting cell population to be >99% CD14⁺. Human monocytes were stimulated with LPS in the presence of mock or rhesus CMV IL-10 conditioned medium. After 48 hours, supernatants were assayed for GM-CSF by ELISA, as described above. In LPS-stimulated monocyte cultures, robust production of GM-CSF was observed. In the presence of rhesus CMV IL-10 the level of GM-CSF was greatly reduced. (See FIG. 7B).

### Example 11

### Effect of Rhesus CMV IL-10 on GM-CSF Synthesis by Human Monocytes is Comparable to the Effect of Recombinant hIL-10 on GM-CSF Synthesis by Human Monocytes

Primary monocytes were purified from human PBMCs as described above. Flow cytometric analysis was performed as described above and revealed the resulting cell population to be >99% CD14⁺. Human monocytes were stimulated with LPS in the presence of mock or recombinant hIL-10 conditioned medium. After 48 hours, supernatants were assayed for GM-CSF by ELISA as described above. In LPS-stimulated monocyte cultures, robust production of GM-CSF was observed. The decrease in GM-CSF levels in cultures containing hIL-10 were comparable to the decrease in GM-CSF levels in cultures containing rhesus CMV IL-10. (See FIG. 7B).

### Example 12

### Rhesus CMV IL-10 Inhibits IL-1α Synthesis by Human Monocytes

Primary monocytes were purified from human PBMCs as described above. Flow cytometric analysis was performed as described above and revealed the resulting cell population to be >99% CD14⁺. Human monocytes were stimulated with LPS in the presence of mock or rhesus CMV IL-10 conditioned medium. After 48 hours, supernatants were assayed for IL-1α by ELISA, as described above. In LPS-stimulated monocyte cultures, robust production of IL-1α was observed. In the presence of rhesus CMV IL-10 the level of IL-1α was greatly reduced. (See FIG. 7A).

### Example 13

### Effect of Rhesus CMV IL-10 IL-1α Synthesis by Human Monocytes is Comparable to the Effect of Recombinant hIL-10 on IL-1α Synthesis by Human Monocytes

Primary monocytes were purified from human PBMCs as described above. Flow cytometric analysis was performed as described above and revealed the resulting cell population to be >99% CD14⁺. Human monocytes were stimulated with LPS in the presence of mock or recombinant hIL-10 conditioned medium. After 48 hours, supernatants were assayed for IL-1α by ELISA as described above. In LPS-stimulated monocyte cultures, robust production of IL-1α was observed. The decrease in IL-1α levels in cultures containing hIL-10 were comparable to the decrease in IL-1α levels in cultures containing rhesus CMV IL-10. (See FIG. 7A).

### Example 14

### Rhesus CMV IL-10 Inhibits IL-6 Synthesis by Human Monocytes

PBMCs were obtained from two human donors. Primary monocytes were purified from the PBMCs, as described above. Flow cytometric analysis was performed as described above and revealed the resulting cell population to be >99% CD14⁺. Human monocytes were stimulated with LPS in the presence of mock or rhesus CMV IL-10 conditioned medium. After 48 hours, supernatants were assayed for IL-6 by ELISA, as described above. In LPS-stimulated monocyte cultures, robust production of IL-6 was observed. In the presence of rhesus CMV IL-10 the level of IL-6 was greatly reduced. (See FIG. 7D).

### Example 15

### Rhesus CMV IL-10 Reduces Surface Expression of Classical Class I MHC Molecules by Monocytes

Primary monocytes were purified from human PBMCs as described above. LPS stimulated human monocytes cultured in the presence of mock or rhesus CMV IL-10 conditioned media. After 48 hours, the cells were stained with antibodies to classical class I MHC molecules and analyzed by flow cytometry, as described above. Cell surface levels of classical class I MHC molecules were reduced by incubation with rhesus CMV IL-10. (See FIG. 9, HLA-A, B, C panels). In FIG. 9, the solid curve represents antibody staining and open curve represents antibody staining with the isotype controls. Decreased cell surface expression of class I MHC molecules, measured by the mean fluorescence intensity of class I MHC staining as described above, was observed in 4/6 donors, while 2/6 donors were unaffected.

### Example 16

### Rhesus CMV IL-10 Reduces Surface Expression of Class II MHC Molecules by Monocytes

Primary monocytes were purified from human PBMCs as described above. LPS stimulated human monocytes cultured in the presence of mock or rhesus CMV IL-10 conditioned media. After 48 hours, the cells were stained with antibodies to the class II MHC molecule and analyzed by flow cytometry, as described above. Cell surface levels of class II MHC molecules were reduced by incubation with rhesus CMV IL-10. (See FIG. 9, HLA-DR panel). Levels of class II MHC molecules were decreased by rhesus CMV IL-10 treatment in 6/6 donors tested. The mean fluorescence intensity of class II MHC staining for rhesus CMV IL-10 treated cells was at least three-fold lower than cells treated with the mock conditioned medium for all donors.

### Example 17

### Rhesus CMV IL-10 Increases Surface Expression of HLA-G, a Nonclassical Class I MHC Molecule, by Monocytes

Primary monocytes were purified from human PBMCs as described above. LPS stimulated monocytes cultured in the presence of mock or rhesus CMV IL-10 conditioned media. After 48 hours, the cells were stained with antibodies to HLA-G and analyzed by flow cytometry, as described above. Cell surface levels of classical HLA-G were increased by incubation with rhesus CMV IL-10. (See FIG. 9, HLA-G panel). Up regulation of cell surface HLA-G expression was seen in all donors tested (6/6). The mean fluorescence intensity of nonclassical class II MHC staining for rhesus CMV IL-10 treated cells was increased approximately two-fold in each case.

### Example 18

### Rhesus CMV IL-10 does not Effect the Surface Expression of CD54 (ICAM) by Monocytes

Primary monocytes were purified from human PBMCs as described above. LPS stimulated monocytes cultured in the presence of mock or rhesus CMV IL-10 conditioned media. After 48 hours, the cells were stained with antibodies to CD54 and analyzed by flow cytometry, as described above. Cell surface expression of CD54 on LPS stimulated monocytes was not significantly effected by rhesus CMV IL-10 treatment. (See FIG. 9, CD54 panels).

### Example 19

### Expression of Recombinant Human CMV IL-10 in Human Embryonic Kidney Cells

Human embryonic kidney (HEK) 293 cells were grown in DMEM (Hyclone) plus 5% FCS. The HEK293 cells were transfected with an expression vector encoding a C-terminal myc/His epitope-tagged human CMV IL-10 protein from human CMV strain Towne. Supernatants were collected after 48 hours, clarified by low speed centrifugation and human CMV IL-10 expression was verified by Western blot with an anti-poly-His monoclonal antibody (Invitrogen). Human CMV IL-10 was detected using TMB Membrane Peroxidase Substrate (Kirkegaard & Perry Laboratories). Transfectant supernatants were harvested and found to express a protein of 28 kD, the predicted size of the epitope-tagged human CMV IL-10 (see Kotenko et al., PNAS 97:4, 1695-1700, 2000) until at least 48 hours post-transfection. Mock conditioned medium was obtained from cells transfected with the control vector (pcDNA3.1-m/H, Invitrogen). (See FIG. 1).

### Example 20

### Human CMV IL-10 Inhibits Proliferation of Human PBMCs

Recombinant human CMV IL-10 was generated as described above. The supernatants, obtained as described above, were used to assay the effects of human CMV IL-10 on human PBMC proliferation in the absence of other virally produced factors. PBMC proliferation was measured as described above. PBMCs from healthy human donors (n=2) were stimulated with PHA for 72 hours in the presence of human CMV IL-10 or mock supernatants.

Each donor, showed marked inhibition of PBMC proliferation in the presence of human CMV IL-10. PHA stimulated human PBMCs exhibited robust proliferation, but this proliferation was inhibited in the presence of human CMV IL-10. (See FIG. 2C). Proliferation of human PBMCs cultured in the presence of the mock conditioned medium was not found to differ from human PBMCs cultured in fresh RPMI.

### Example 21

### Relative Specific Activity of Recombinant Human IL-10 and Recombinant Human CMV IL-10

Freshly isolated human PBMCs were incubated in RPMI with 10% FBS with 5 μg/ml PHA in the presence or absence of recombinant human IL-10 or recombinant human CMV IL-10. At 48 hours, tritiated thymidine was added (40 Ci/ml) and well were harvested after 18 hours incubation, Incorporated tritium was quantitated on a scintillation counter. Both the recombinant human IL-10 and the recombinant human CMV IL-10 inhibited PHA induced proliferation of PBMCs. (See FIGS. 22A and 22B, respectively). Recombinant human IL-10 and the recombinant human CMV IL-10 were tested over a broad concentration titration, ranging from 100 ng/ml to 1 pg/ml final concentration. Results indicate both recombinant human IL-10 and recombinant human CMV IL-10 inhibited proliferation of PHA stimulated human PBMCs in a dose dependent manner. (See FIGS. 11A and 11B, respectively). Doses of recombinant human IL-10 or recombinant human CMV IL-10 as low as 10 pg/ml significantly inhibited the human PBMC proliferative response.

### Example 22

### Neutralizing Monoclonal Antibody to Human IL-10 Receptor Reverses the Ability of Recombinant Human CMV IL-10 to Inhibit the Proliferation of PHA Stimulated Human PBMCs

Freshly isolated human PBMCs were incubated in RPMI with 10% FBS with 10 μ/ml PHA in the presence or absence of recombinant human IL-10 or recombinant human CMV IL-10. At 48 hours, tritiated thymidine was added (40 Ci/ml) and well were harvested after 18 hours incubation. Incorporated tritium was quantitated on a scintillation counter. Recombinant human IL-10 and recombinant human CMV IL-10 inhibited PHA induced proliferation of PBMCs. A 15 μg/ml of a monoclonal antibody specific for the human IL-10 receptor was added to the samples. Addition of the antibody reduced the activity of both the recombinant human IL-10 or recombinant human CMV IL-10. (See FIGS. 12A and 12B, respectively).

### Example 23

### Human CMV IL-10 Inhibits IFN-γ Synthesis by Human PBMCs

PBMCs were obtained from four human donors. The PBMCs were stimulated with PHA and cultured in the presence of mock or human CMV IL-10 conditioned medium. After 48 hours, supernatant IFN-γ levels were assayed by ELISA as described above. While control human PBMC cultures produced IFN-γ in response to PHA stimulation, the levels IFN-γ were decreased in cultures containing human CMV IL-10. Production of IFN-γ was almost completely abrogated in the presence of human CMV IL-10. (See FIG. 5B and 5C).

### Example 24

### Effect of Human CMV IL-10 on IFN-γ Synthesis by Human PBMCs is Comparable to the Effect of Recombinant hIL-10 on IFN-γ Synthesis by Human PBMCs

PHA-stimulated human PBMCs were cultured in the presence of mock or 1 μg/ml recombinant hIL-10 conditioned medium. After 48 hours, supernatant IFN-γ levels were assayed by ELISA as described above. While control human PBMC cultures produced IFN-γ in response to PHA stimulation, the levels IFN-γ were decreased in cultures containing hIL-10. The decrease in IFN-γ levels in cultures containing hIL-10 were comparable to the decrease in IFN-γ levels in cultures containing human CMV IL-10. (See FIGS. 5B and 5C).

### Example 25

### Human CMV IL-10 Inhibits TNF-α Synthesis by Human PBMCs

PHA-stimulated human PBMCs were cultured in the presence of mock or human CMV IL-10 conditioned medium. After 48 hours, supernatant TNF-α levels were assayed by ELISA as described above. While control human PBMC cultures produced TNF-α in response to PHA stimulation, the levels TNF-α were decreased in cultures containing human CMV IL-10 (n=2). Production of TNF-α was almost completely abrogated in the presence of human CMV IL-10. (See FIG. 6B).

### Example 26

### Effect of Human CMV IL-10 on TNF-α Synthesis by Human PBMCs is Comparable to the Effect of Recombinant hIL-10 on TNF-α Synthesis by Human PBMCs

PHA-stimulated human PBMCs were cultured in the presence of mock or 1 μg/ml recombinant hIL-10 conditioned medium. After 48 hours, supernatant TNF-α levels were assayed by ELISA as described above. While control human PBMC cultures produced TNF-α in response to PHA stimulation, the levels TNF-α were decreased in cultures containing hIL-10. The decrease in TNF-α levels in cultures containing hIL-10 were comparable to the decrease in TNF-α levels in cultures containing human CMV IL-10. (See FIG. 6B).

### Example 27

### Human CMV IL-10 Inhibits TNF-α Synthesis by Human Monocytes

Primary monocytes were purified from four human donors of PBMCs as described above. Flow cytometric analysis was performed as described above and revealed the resulting cell population to be >99% CD14⁺. Human monocytes were stimulated with LPS in the presence of mock or human CMV IL-10 conditioned medium. After 48 hours, supernatants were assayed for TNF-α by ELISA, as described above. In LPS-stimulated monocyte cultures, robust production of TNF-α was observed. In the presence of human CMV IL-10 the level of TNF-α was greatly reduced. (See FIG. 8A).

### Example 28

### Effect of Human CMV IL-10 on TNF-α Synthesis by Human PBMCs is Comparable to the Effect of Recombinant hIL-10 on TNF-α Synthesis by Human Monocytes

Primary monocytes were purified from human PBMCs as described above. Flow cytometric analysis was performed as described above and revealed the resulting cell population to be >99% CD14⁺. Human monocytes were stimulated with LPS in the presence of mock or recombinant hIL-10 conditioned medium. After 48 hours, supernatants were assayed for TNF-α by ELISA as described above. In LPS-stimulated monocyte cultures, robust production of TNF-α was observed. The decrease in TNF-α levels in cultures containing hIL-10 were comparable to the decrease in TNF-α levels in cultures containing human CMV IL-10. (See FIG. 8A).

### Example 29

### Human CMV IL-10 Inhibits GM-CSF Synthesis by Human Monocytes

Primary monocytes were purified, described above, from the PBMCs of four human donors. Flow cytometric analysis was performed as described above and revealed the resulting cell population to be >99% CD14⁺. Human monocytes were stimulated with LPS in the presence of mock or human CMV IL-10 conditioned medium. After 48 hours, supernatants were assayed for GM-CSF by ELISA, as described above. In LPS-stimulated monocyte cultures, robust production of GM-CSF was observed. In the presence of human CMV IL-10 the level of GM-CSF was greatly reduced. (See FIG. 8B).

### Example 30

### Effect of Human CMV IL-10 on GM-CSF Synthesis by Human PBMCs is Comparable to the Effect of Recombinant hIL-10 on GM-CSF Synthesis by Human Monocytes

Primary monocytes were purified from human PBMCs as described above. Flow cytometric analysis was performed as described above and revealed the resulting cell population to be >99% CD14⁺. Human monocytes were stimulated with LPS in the presence of mock or recombinant hIL-10 conditioned medium. After 48 hours, supernatants were assayed for GM-CSF by ELISA as described above. In LPS-stimulated monocyte cultures, robust production of GM-CSF was observed. The decrease in GM-CSF levels in cultures containing hIL-10 were comparable to the decrease in GM-CSF levels in cultures containing human CMV IL-10. (See FIG. 8B).

### Example 31

### Human CMV IL-10 Inhibits IL-1α Synthesis by Human Monocytes

PBMCs were obtained from two human donors. Primary monocytes were purified from the PBMCs, as described above. Flow cytometric analysis was performed as described above and revealed the resulting cell population to be >99% CD14⁺. Human monocytes were stimulated with LPS in the presence of mock or human CMV IL-10 conditioned medium. After 48 hours, supernatants were assayed for IL-1α by ELISA, as described above. In LPS-stimulated monocyte cultures, robust production of IL-1α was observed. In the presence of human CMV IL-10 the level of IL-1α was greatly reduced. (See FIG. 8C).

### Example 32

### Effect of Human CMV IL-10 on IL-1α Synthesis by Human Monocytes is Comparable to the Effect of Recombinant hIL-10 on IL-1α Synthesis by Human Monocytes

Primary monocytes were purified from human PBMCs as described above. Flow cytometric analysis was performed as described above and revealed the resulting cell population to be >99% CD14⁺. Human monocytes were stimulated with LPS in the presence of mock or recombinant hIL-10 conditioned medium. After 48 hours, supernatants were assayed for IL-1α by ELISA as described above. In LPS-stimulated monocyte cultures, robust production of IL-1α was observed. The decrease in IL-1α levels in cultures containing hIL-10 were comparable to the decrease in IL-1α levels in cultures containing human CMV IL-10. (See FIG. 8C).

### Example 33

### Human CMV IL-10 Inhibits IL-6 Synthesis by Human Monocytes

PBMCs were obtained from two human donors. Primary monocytes were purified from the PBMCs, as described above. Flow cytometric analysis was performed as described above and revealed the resulting cell population to be >99% CD14⁺. Human monocytes were stimulated with LPS in the presence of mock or human CMV IL-10 conditioned medium. After 48 hours, supernatants were assayed for IL-6 by ELISA, as described above. In LPS-stimulated monocyte cultures, robust production of IL-6 was observed. In the presence of human CMV IL-10 the level of IL-6 was greatly reduced. (See FIG. 8D).

### Example 34

### Human CMV IL-10 Reduces Surface Expression of Classical Class I MHC Molecules by Monocytes

Primary monocytes were purified from human PBMCs as described above. LPS stimulated human monocytes cultured in the presence of mock or human CMV IL-10 conditioned media. After 48 hours, the cells were stained with antibodies to classical class I MHC molecules and analyzed by flow cytometry, as described above. Cell surface levels of classical class I MHC molecules were reduced by incubation with human CMV IL-10. (See FIG. 10, HLA-A panel). In FIG. 10, the solid curve represents antibody staining and open curve represents antibody staining with the isotype controls. Decreased cell surface expression of class I MHC molecules, measured by the mean fluorescence intensity of class I MHC staining as described above, was observed in 2/4 donors, while 2/4 donors were unaffected.

### Example 35

### Human CMV IL-10 Reduces Surface Expression of Class II MHC Molecules by Monocytes

Primary monocytes were purified from human PBMCs as described above. LPS stimulated human monocytes cultured in the presence of mock or human CMV IL-10 conditioned media. After 48 hours, the cells were stained with antibodies to the class II MHC molecule and analyzed by flow cytometry, as described above. Cell surface levels of class II MHC molecules were reduced by incubation with human CMV IL-10. (See FIG. 10, HLA-DR panel). Levels of class II MHC molecules were decreased by human CMV IL-10 treatment in 3/4 donors tested, while 1/4 donors were unaffected.

### Example 36

### Human CMV IL-10 does not Effect the Surface Expression of CD54 by Monocytes

Primary monocytes were purified from human PBMCs as described above. LPS stimulated monocytes cultured in the presence of mock or human CMV IL-10 conditioned media. After 48 hours, the cells were stained with antibodies to CD54 and analyzed by flow cytometry, as described above. Cell surface expression of CD54 on LPS stimulated monocytes was not significantly effected by human CMV IL-10 treatment. (See FIG. 10, CD54 panels).

### Example 37

### Murine Model for Endotoxin-Induced Shock

Five groups of six mice are primed with 0.5 mg heat-killed C. parvum, administered intravenous (i.v.), as a challenge to induce a condition resembling septic shock As a control, a first group is treated with phosphate buffered saline (PBS) and Tween 20 0.5% one hour before challenge. As a further control, the first group is treated with mouse serum albumin (MSA, a protein placebo) at the time of challenge. A second group is treated with 0.1 mg/KG betamethasone phosphate in buffered saline p.o. one hour before challenge. A third group is treated with rhesus CMV IL-10 administered i.p. at time of challenge. A fourth group is treated with human CMV IL-10 administered i.p. at time of challenge.

Ninety minutes, and at various times thereafter, post challenge blood is drawn from each mouse and tested for the concentrations of IL-1 and TNF-α. Efficacy of the treatment may be monitored by testing for concentrations of IL-1 and TNF-α.

### Example 38

### Murine Model for Endotoxin-Induced Shock

Three groups of six mice are primed with a dose of TNF-α sufficient to induce a condition resembling septic shock. As a control, a first group is treated with phosphate buffered saline (PBS) and Tween 20 0.5% one hour before challenge. As a further control, the first group is treated with mouse serum albumin (MSA, a protein placebo) at the time of challenge. A third group is treated with rhesus CMV IL-10 administered intraperitoneal (i.p.) at time of challenge. A fourth group is treated with human CMV IL-10 administered i.p. at time of challenge.

Ninety minutes, and at various times thereafter, post challenge blood is drawn from each mouse and tested for the concentrations of IL-1 and TNF-α. Efficacy of the treatment may be monitored by testing for concentrations of IL-1 and TNF-α.

### Example 39

### Murine Model for Lethal Endotoxin-Induced Shock

Five groups of 15 mice are selected from a mouse colony, and are primed with an i.p. injection of an LD₉₀ dose of lipopolysaccharide (LPS) as a challenge. The precise LD₉₀ dose of LPS is determined by titration of the LPS batch in the mouse colony. As a control, a first group of mice additionally receive phosphate buffered saline (PBS) and Tween 0.5% administered i.p. 30 minutes before the LPS challenge. A second group is treated with rhesus CMV IL-10 administered i.p. 30 minutes before the LPS challenge. A third group is treated with human CMV IL-10 administered i.p. 30 minutes before the LPS challenge. A fourth group is treated with human IL-10 administered i.p. 30 minutes before the LPS challenge. A fifth group is treated with mouse IL-10 administered i.p. 30 minutes before LPS the challenge.

Mice are monitored for death over the 72 hours following LPS challenge. Mouse survival time, post-LPS challenge, is a measure of treatment efficacy. Efficacy of the treatment may be also monitored by testing for concentrations of IL-1 and TNF-α.

### Example 40

### Murine Model for Graft Verses-Host Disease

Immunocompetent lymphocytes are introduced into three groups of six mice. As a control, phosphate buffered saline (PBS) and Tween 20 0.5%, lymphocytes that are not immunocompetent are introduced into a fourth group of mice. All of the mice are either allogeneic neonates or x-irradiated mice. The grafted lymphocytes are carried to the spleen, where they begin to proliferate in response to the allogenetic MHC antigens of the recipient. This proliferation induces an influx of recipient cells and results in splenomegaly. The intensity of GVHD can be quantitated by calculating the spleen index as follows:

$$Spleen index = \frac{weight of experimental spleen / total body weight}{weight of control spleen / total body weight}$$

A spleen index of 1.3 or greater is considered to indicative of GVHD.

At predetermined times, the first group is treated with rhesus CMV IL-10 administered i.p., the second group is treated with human CMV IL-10 administered i.p. and, as controls, the third and fourth groups are treated with mouse serum albumin (MSA, a protein placebo).

At predetermined times, the mice of each group are sacrificed and their SI determined. The SI is a measure of treatment efficacy.

### Example 41

### Rat Model for Type 1 Hypersensitivity

Five groups of 10 rats are actively sensitized on Day 0 (initial sensitization) by a single i.p. injection of 100 μg OVA in phosphate-buffered saline (PBS), along with an IgE-selective adjuvant, e.g., aluminum hydroxide. At the peak of the rat IgE response, e.g., Day 11, the rats are placed in a Plexiglas chamber and challenged with aerosolized OVA (1%) for 30 minutes using an ultrasonic nebulizer (De Vilbliss). One group of rats additionally receive phosphate buffered saline (PBS)and Tween 0.5% i.p. at the initial sensitization, and at different dosing schedules thereafter, up until the aerosolized OVA challenge. A second group is treated with rhesus CMV IL-10 administered i.p. at the initial sensitization, and at different dosing schedules thereafter, up until the aerosolized OVA challenge. A third group is treated with human CMV IL-10 administered i.p. at the initial sensitization, and at different dosing schedules thereafter, up until the aerosolized OVA challenge. A fourth group is treated with human IL-10 administered i.p. at the initial sensitization, and at different dosing schedules thereafter, up until the aerosolized OVA challenge. A fifth group is treated with rat IL-10 administered i.p. at the initial sensitization, and at different dosing schedules thereafter, up until the aerosolized OVA challenge.

Efficacy of treatment may be monitored by testing pulmonary function, cellular infiltrates in bronchoalveolar lavage (BAL), histological examination of lungs, and serum OVA-specific IgE titers of the rats at different time points post aerosolized OVA challenge. Efficacy of treatment may also be monitored by testing levels of TNF-α, IL-6, and INF-γ.

### Example 42

### Murine Model for Delayed Hypersensitivity (Type IV) Reaction

Ten groups of 15 mice are infected (initial sensitization) with the intracellular parasite Leishmania major (L. major) by injecting L. major promastigotes subcutaneously into their left hind footpads. The first and sixth groups of mice additionally receive phosphate buffered saline (PBS)and Tween 0.5% i.p. at the initial sensitization, and at different dosing schedules thereafter, up until the Leishmania antigen challenge. The second and seventh groups are treated with rhesus CMV IL-10 administered i.p. at the initial sensitization, and at different dosing schedules thereafter, up until the Leishmania antigen challenge. The third and eighth groups are treated with human CMV IL-10 administered i.p. at the initial sensitization, and at different dosing schedules thereafter, up until the Leishmania antigen challenge. The fourth and ninth groups are treated with human IL-10 administered i.p. at the initial sensitization, and at different dosing schedules thereafter, up until the Leishmania antigen challenge. The fifth and tenth groups are treated with mouse IL-10 administered i.p. at the initial sensitization, and at different dosing schedules thereafter, up until the Leishmania antigen challenge. Four weeks after infection, the first, second, third, fourth, and fifth groups of mice are challenged, in the contra-lateral footpad, with Leishmania freeze-thawed antigen; and, groups six, seven, eight, nine, and ten are treated, in the contra-lateral footpad, with PBS as a negative control.

Efficacy of treatment may be monitored by measuring footpad swelling with a metric caliper. Efficacy of treatment may also be monitored by measuring the proliferation, cytokine production, and other phenotypic criteria of draining lymph node T cells.

### Example 43

### Murine Model for Rheumatoid Arthritis

Five groups of mice genetically-susceptible to rheumatoid arthritis are injected subcutaneously or intra-dermal with type II collagen emulsified in Complete Freund's Adjuvant at Day 0 (initial sensitization) and Day 21. A first group of mice additionally receive phosphate buffered saline (PBS)and Tween 0.5% administered i.p. at the initial sensitization, and at different dosing schedules thereafter. A second group is treated with rhesus CMV IL-10 administered i.p. at the initial sensitization, and at different dosing schedules thereafter. A third group is treated with human CMV IL-10 administered i.p. at the initial sensitization, and at different dosing schedules thereafter. A fourth group is treated with human IL-10 administered i.p. at the initial sensitization, and at different dosing schedules thereafter. A fifth group is treated with mouse IL-10 administered i.p. at the initial sensitization, and at different dosing schedules thereafter.

From weeks 3 to 8, the mice are monitored for the development of swollen joints or paws, and graded on a standard disease severity scale. Disease severity is confirmed by histological analysis of joints. Efficacy of treatment is monitored by the disease severity as measured by the development of swollen joint or paws. Efficacy of treatment may also measured by histological analysis of joint.

### Example 44

### Murine Model for Inflammatory Bowel Disease

Five groups of mice having of inflammatory bowel disease (IBD) are tested for rhesus CMV IL-10 or human CMV IL-10 efficacy as follows. The first group, a control group, of mice are treated with phosphate buffered saline (PBS) and Tween 0.5% administered i.p., and are treated with varying dosings thereafter. A second group is treated with rhesus CMV IL-10 administered i.p., and are treated with varying dosings thereafter. A third group is treated with human CMV IL-10 administered i.p., and are treated with and varying dosings thereafter. A fourth group is treated with human IL-10 administered i.p., and are treated with varying dosings thereafter. A fifth group is treated with mouse IL-10 administered i.p., and treated with varying dosings thereafter.

Mice are monitored for 6-8 weeks during which time IBD may develop. The progress of disease is monitored initially via weight loss and/or prolapsed rectum, and eventually by histological evaluation of the colon and intestinal tract. Efficacy of rhesus or human CMV IL-10 treatment may be monitored by comparing the progress of disease in the mice of group one to the mice of groups two and three, respectively.

It would be apparent to one of skill in the art would that many mouse models of IBD are appropriate to test for rhesus CMV IL-10 or human CMV IL-10 efficacy. A spontaneous model of IBD is provided by in transgenic mice depleted of certain cytokine genes (e.g., IL-10, or IL-2). In the spontaneous mouse model, the first treatment in all groups of mice occurs soon after weaning. A cell transfer mouse model of IBD is created by transferring highly purified populations of CD4+ T lymphocytes bearing a particular surface marker phenotype (e.g., CD45 RB hi) into SCID mice. In the cell transfer mouse model, the first treatment in all groups of mice occurs at time of cell transfer into the SCID mice.

### Example 45

### Murine Model for Systemic Lupus Erythematous

Commencing at 6 months of age, female NZB/W F1 mice spontaneously develop an SLE-like pathology characterized by proteinuria, serum autoantibodies, glomerulonephritis, and eventually death. Five groups of 20 NZB/W mice are tested for rhesus or human CMV IL-10 efficacy as follows. A first group, a control group, of mice are treated with receives phosphate buffered saline (PBS) and Tween 0.5% i.p. soon after weaning, and thereafter at varying dosing schedules. A second group of 20 NZB/W mice are treated with rhesus CMV IL-10 administered i.p. soon after weaning, and thereafter at varying dosing schedules. A third group of 20 NZB/W mice are treated with human CMV IL-10 administered i.p. soon after weaning, and thereafter at varying dosing schedules. A fourth group of 20 NZB/W mice are treated with human IL-10 administered i.p. soon after weaning, and thereafter at varying dosing. A fifth group of 20 NZB/V mice are treated with mouse IL-10 administered i.p. soon after weaning, and thereafter at varying dosing.

Efficacy of treatment may be monitored by analyzing kidney histology, measuring serum autoantibody levels, and detecting proteinuria. Efficacy of treatment may also be measured in terms of mortality.

### Example 46

### Murine Model for Tumor Growth

Five groups of a mouse model of tumor growth are tested for rhesus CMV IL-10 or human CMV IL-10 efficacy as follows. A first group, a control group, is treated with phosphate buffered saline (PBS) and Tween 0.5% administered i.p. soon after tumor transplant, and thereafter at varying dosing schedules. A second group is treated with rhesus CMV IL-10 administered i.p. soon after tumor transplant, and thereafter at varying dosing schedules. A third group is treated with human CMV IL-10 administered i.p. soon after tumor transplant, and thereafter at varying dosing schedules. A fourth group is treated with human IL-10 administered i.p. soon after tumor transplant, and thereafter at varying dosing. A fifth group is treated with mouse IL-10 administered i.p. soon after tumor transplant, and thereafter at varying dosing.

It would be apparent to one of skill in the art would that many mouse models of tumor growth are appropriate to test for rhesus CMV IL-10 or human CMV IL-10 efficacy. SCID mice can be transplanted with primary human tumor cells. Normal mouse strains can be transplanted with a variety of well-characterized mouse tumor lines, including a mouse thymoma EL4 which has been transfected with OVA to allow easy evaluation of tumor specific antigen responses.

Efficacy of rhesus or human CMV IL-10 treatment may be monitored via tumor growth versus regression. In the case of the OVA-transfected EL4 thymoma mouse model of tumor growth, cytolytic OVA-specific responses can be measured by stimulating draining lymph node cells with OVA in vitro, and measuring antigen-specific cytotoxicity at 72 hours.

Although the foregoing invention has been described in detail for purposes of clarity of understanding, it will be obvious that certain modifications may be practiced within the scope of the appended claims. All publications and patent documents cited herein are hereby incorporated by reference in their entirety for all purposes to the same extent as if each were so individually denoted.

What is claimed is:

1. A method of inhibiting the proliferation of a peripheral blood mononuclear cell population, comprising contacting the peripheral blood mononuclear cell population with an amount of rhesus or human CMV IL-10 sufficient to inhibit the proliferation of the peripheral blood mononuclear cell population.

2. The method of claim 1, wherein the peripheral blood mononuclear population is contacted with rhesus CMV IL-10.

3. The method of claim 1, wherein the peripheral blood mononuclear population is contacted with human CMV IL-10.

4. The method of claim 1, wherein peripheral blood mononuclear, cells are proliferating when the contacting step is performed.

5. The method of claim 1, wherein the contacting occurs in vitro.

6. The method of claim 1, further comprising adding an agent that induces the peripheral blood mononuclear cells to proliferate.

7. The method of claim 1, wherein the level of IFN-γ secreted by the peripheral blood mononuclear is cells is detectably reduced responsive to the contacting step.

8. The method of claim 1, wherein the level of TNF-α secreted by the peripheral blood monocular cells is detectably reduced responsive to the contacting step.

9. The method of claim 1, further comprising monitoring the proliferation level of the peripheral blood mononuclear cells to determine a reduction in the proliferation level responsive to the contacting step.

10. The method of claim 1, further comprising monitoring secretion of IFN-γ or TNF-α to determine a reduction in level of secreted IFN-γ or TNF-α responsive to the contacting step.

11. The method of claim 1, wherein the mononuclear proliferating cells are rhesus or human cells.

12. A method of reducing cytokine production of a monocyte cell population, comprising contacting the monocyte cell population with an amount of rhesus or human CMV IL-10 sufficient to reduce cytokine production by the monocyte cell population.

13. The method of claim 12, wherein the contacting occurs in vitro.

14. The method of claim 12, wherein the level of IFN-γ secreted by the monocytes is detectably reduced responsive to the contacting step.

15. The method of claim 12, wherein the level of TNF-α secreted by the monocytes is detectably reduced responsive to the contacting step.

16. The method of claim 12, wherein the level of GM-CSF secreted by the monocytes is detectably reduced responsive to the contacting step.

17. The method of claim 12, wherein the level of IL-1α secreted by the monocytes is detectably reduced responsive to the contacting step.

18. The method of claim 12, wherein the level of IL-6 secreted by the monocytes is detectably reduced responsive to the contacting step.

19. The method of claim 12, further comprising monitoring the cytokine levels of the monocytes to determine a reduction in the proliferation level responsive to the contacting step.

20. The method of claim 12, further comprising monitoring secretion of IFNγ, TNF-α, GM-CSF, IL-1α or IL-6 to determine a reduction in level of secreted IFN-γ, TNF-α, GM-CSF, IL-1α or IL-6, responsive to the contacting step.

21. A method of preventing or treating an immune disorder in a patient, comprising:
administering rhesus CMV IL-10 or human CMV IL-10 to a patient suffering from or susceptible to the disorder in a dosage sufficient to inhibit proliferation of lymphocytes in the patient, and thereby prevent or treat the disorder.

22. The method of claim 21, wherein the rhesus CMV IL-10 or human CMV IL-10 is a component of a pharmaceutical composition further comprising a pharmaceutically acceptable carrier.

23. The method of claim 21, wherein the pharmaceutical composition is sterile, substantially isotonic and prepared under GMP conditions.

24. The method of claim 21, wherein the patient is suffering from or susceptible to an immune disorder selected from the group consisting of graft versus host disease, an autoimmune disease, an inflammatory response, a pathologic delayed type hypersensitivity response, endotoxin-induced toxic shock, granulomatis disease, psoriasis, uveitis, systemic lupus erythematous, multiple sclerosis and contact-dermatitis.

25. The method of claim 21, further comprising monitoring proliferation of the lymphocytes in the patient to detect a reduction in the level of proliferation responsive to the administering step.

26. The method of claim 21, further comprising monitoring a symptom of the patient, to detect amelioration or prevention of the symptom responsive to the administering step.

27. The method of claim 21, wherein the patient is suffering from the disorder.

28. The method of claim 21, wherein the patient is susceptible to the disorder.

29. The method of claim 28, wherein the patient is an organ transplant patient.

30. The method of claim 29, wherein the organ is a kidney.

31. The method of claim 30, wherein the IFN-α levels are detectably decreased responsive to the administering of rhesus or human CMV IL-10.

32. The method of claim 21, wherein the inflammatory disorder is a chronic inflammatory response.

33. The method of claim 32 wherein the chronic inflammatory disease is selected from the group consisting of rheumatoid arthritis, inflammatory bowel disease, Crohn's disease, ulcerative colitis, Graves' disease, Hashimoto's thyroiditis, systemic lupus erythematosus, multiple sclerosis, scleroderma, and insulin-dependent diabetes mellitus.

34. The method of claim 21, wherein the inflammatory disorder is an allergic response.

35. The method of claim 34, wherein the inflammatory disorder is asthma.

36. The method of claim 21, wherein the patient is suffering from a type T_{H}1 immune response to transplanted graft.

37. The method of claim 36, wherein the transplanted graft is an organ selected from the group consisting of cornea, lung, heart, liver, bone marrow, kidney, pancreas, blood, and skin.

38. The method of claim 25 wherein the immune disorder is leukemia.

39. A method of ameliorating symptoms of hepatitis in an animal host, comprising administering to the animal infected with hepatitis virus an effective dosage CMV IL-10 sufficient to ameliorate at least one of the symptoms of hepatitis.

40. The method of claim 39, wherein the administering step ameliorates damage liver in the patient.

41. The method of claim 39, wherein the administering step ameliorates liver disease or liver fibrosis.

42. A method of treating or preventing a respiratory viral infection in a patient, comprising administering rhesus or human CMV IL-10 to the patient suffering from or susceptible to a virally infected respiratory system in a dosage sufficient to ameliorate at least one symptom of the respiratory viral infection.

43. A method for reducing an in vivo inflammatory response characterized by substantially elevated levels of at least one cytokine selected from the group consisting of IL-1α, GM-CSF, IFN-γ and TNF-α, comprising administering to the patient afflicted with such an inflammatory response or at risk for developing such an inflammatory response, an effective dosage of rhesus CMV IL-10 or human CMV IL-10 to substantially lower the levels of said cytokines.

44. A method of preventing or treating the symptoms of an inflammatory response, comprising administering rhesus CMV IL-10 or human CMV IL-10 to the patient suffering from or susceptible to an inflammatory response in a dosage sufficient to ameliorate at least some of the symptoms of the inflammatory condition.

45. The method of claim 44, further comprising monitoring proliferation of the lymphocytes in the patient to detect a reduction in the level of proliferation responsive to the administering step.

46. The method of claim 44, further comprising monitoring a symptom of the patient, to detect amelioration or prevention of the symptom responsive to the administering step.

47. The method of claim 44, wherein the patient is suffering from the disorder.

48. The method of claim 44 wherein the inflammatory response is a chronic inflammatory response.

49. The method of claim 48 wherein the chronic inflammatory disease is selected from the group consisting of rheumatoid arthritis, Crohn's disease, ulcerative colitis, Graves' disease, Hashimoto's thyroiditis and insulin-dependent diabetes mellitus.
